(19) United States
(12) Patent Application Publication
(10) Pub. No.: US 2017/0007349 A1
(43) Pub. Date: Jan. 12, 2017

(54) TRAJECTORY GUIDE, ACCESS PORT, AND FIDUCIAL MARKER ALIGNMENT

(71) Applicants: MATTHEW S. SOLAR, INDIALANTIC, FL (US); CRAIG J. PAGAN, WEST MELBOURNE, FL (US); GLENN D. PERRY, MELBOURNE, FL (US)

(72) Inventors: MATTHEW S. SOLAR, INDIALANTIC, FL (US); CRAIG J. PAGAN, WEST MELBOURNE, FL (US); GLENN D. PERRY, MELBOURNE, FL (US)

(21) Appl. No.: 15/269,675

(22) Filed: Sep. 19, 2016

Related U.S. Application Data
(63) Continuation-in-part of application No. 14/293,168, filed on Jun. 2, 2014, now Pat. No. 9,445,793.
Application No. 14/293,168 is a continuation of application No. 13/707,110, filed on Dec. 6, 2012, now Pat. No. 8,747,419.
Application No. 13/707,110 is a continuation-in-part of application No. PCT/US2011/039963, filed on Jun. 10, 2011.
Application No. PCT/US2011/039963 is a continuation-in-part of application No. 14/826,849, filed on Aug. 14, 2015.
(60) Provisional application No. 61/354,278, filed on Jun. 14, 2010.
(60) Provisional application No. 61/353,251, filed on Jun. 10, 2010.
(60) Provisional application No. 62/117,740, filed on Feb. 18, 2015.
(60) Provisional application No. 62/037,173, filed on Aug. 14, 2014.

(51) Int. Cl. A61B 90/11; A61B 34/20; A61B 17/34

(52) U.S. Cl. CPC A61B 90/11; A61B 17/3421; A61B 34/20; A61B 2017/3407

(57) ABSTRACT

A trajectory guide for introducing an instrument into a human or animal subject is described. A guide stem can be removed in sections without disturbing the aligned instrument. An access port portion of the trajectory guide can be left in place, without disturbing trajectory alignment, and can allow overlying skin to be sutured closed. The access port can provide infusate delivery, such as using an injection port, catheter or the like. A fiducial marker arrangement can provide easy and accurate trajectory alignment, for use with the present trajectory guide, another trajectory guide, or without any trajectory guide.

### CLAIM OF PRIORITY

This patent application is a continuation-in-part of U.S. patent application Ser. No. 14/293,168, filed Jun. 2, 2014, which is a continuation of U.S. patent application Ser. No. 13/707,110, filed on Dec. 6, 2012, which is a continuation-in-part of PCT/US2011/039963, filed on Jun. 10, 2011 (later published as WO 2011/156701A2), through which the present patent application also claims the benefit of priority to: (1) Matthew S. Solar et al. U.S. Provisional Patent Application Ser. No. 61/353,251, entitled “CRANIAL ACCESS PORT DEVICE AND METHOD,” filed on Jun. 10, 2010; and (2) Matthew S. Solar et al. U.S. Provisional Patent Application Ser. No. 61/354,278, entitled “MRI TRAJECTORY GUIDE DEVICE AND METHOD,” filed on Jun. 14, 2010, each of which is hereby incorporated by reference herein in its entirety, and the benefit of priority of each of which is hereby claimed.

This patent application is also a continuation-in-part of U.S. patent application Ser. No. 14/826,849, filed on Aug. 14, 2015, through which the present patent application also claims the benefit of priority to: (1) Matthew S. Solar et al. U.S. Provisional Patent Application Ser. No. 62/037,173, entitled “SKULL-MOUNTED INSTRUMENT TRAJECTORY GUIDE,” filed on Aug. 14, 2014; and (2) Matthew S. Solar et al. U.S. Provisional Patent Application Ser. No. 62/117,740, entitled “SKULL-MOUNTED INSTRUMENT TRAJECTORY GUIDE,” filed on Feb. 18, 2015, each of which is incorporated by reference herein in its entirety, and the benefit of priority of each of which is hereby claimed.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in the drawings that form a part of this document: Copyright 2011, C2C Development, LLC, All Rights Reserved.

### BACKGROUND

A diagnostic, therapeutic, or other interventional procedure on a human or animal subject may involve introducing an instrument toward a desired target location within the subject. For example, an interventional procedure on the subject's brain may involve drilling a burr hole in a subject's skull, mounting a trajectory guide on the subject's skull, and guiding an instrument (e.g., a catheter, a needle, a cannula, an electrode, or other device) to the desired target within the subject, such as by using pre-operative or live images from an imaging modality (e.g., MR, CT, PET, ultrasound, etc.) in an image-guided procedure. Accurate guidance is desirable, particularly for an interventional procedure on the brain, where millimeter or sub-millimeter accuracy of the instrument location may be desirable. Some illustrative examples of interventional procedures on the brain can include, but are not limited to, deep brain stimulation (DBS), infusate delivery (e.g., of a pharmaceutical, biological, or other substance), or microelectrode recording.

Ferrara U.S. Pat. No. 4,809,694 discloses a raised ball-and-socket trajectory guide, in which a deformable ball is located substantially above the burr hole of the skull. In Ferrara, an external thumbscrew can be used to deform the ball to retain an instrument within a ball passage through the deformable ball.

Parmer et al. U.S. Pat. No. 6,902,569 discloses a ball-and-socket trajectory guide with a split ball providing hemispherical sections that capture a relaxable stabilizer. When a guide stem is removed from the ball, the relaxable stabilizer relaxes to grip an instrument within a ball passage through the relaxable stabilizer. After adjusting the instrument trajectory by pivoting the ball, the ball is locked into position using a hexagonal-handled locking member (230) (see Parmer at FIG. 7A that protrudes substantially above the burr hole and the skull). The instrument is then inserted through the locked-in trajectory. The protruding hexagonal-handled locking member (230) is then removed, and a cap 310 is pressed or threaded into place to cover the ball. (See Parmer at col. 14, lines 22-38.)

Skakoon U.S. Pat. No. 7,204,840 and Skakoon U.S. Pat. No. 7,815,861 disclose examples of ball-and-socket trajectory guides that can be used in conjunction with peel-away sheaths. Skakoon U.S. Pat. No. 7,204,840 also shows an example of fiducial markers that can be attached to component associated with a trajectory guide apparatus. (See Skakoon U.S. Pat. No. 7,204,840 at FIG. 39.)

Jenkins U.S. Patent Publication No. 2007/0171184 discloses an example of a raised saddle trajectory guide that can be used in conjunction with a peel-away sheath. (See, e.g., Jenkins at FIG. 6c, [symbol] 0067, [symbol] 0073, [symbol] 0076.)

### Overview

The present inventors have recognized, among other things, that one general problem with certain trajectory guides, for example raised-saddle type trajectory guides, is that they protrude substantially above the burr hole, such that they cannot be left in place chronically after the instrument has been painstakingly delivered to the target in as accurate of a fashion as possible. Such protrusions are not only unappealing in appearance, they can risk injury to the subject in a chronic ambulatory setting, such as if bumped. Instead, such trajectory guides are removed after securing the instrument in place by some other means—but such removal and securing the instrument may itself perturb the location of the instrument.

The present inventors have also recognized that, while a lower-profile ball-and-socket trajectory guide may be used to deliver the instrument to the desired target, many such ball-and-socket trajectory guides still protrude substantially above the burr hole, such that they cannot be left in place chronically after the instrument has been painstakingly delivered to the target in as accurate of a fashion as possible, as explained above. The present inventors have recognized that, for example, a ball-and-socket trajectory guide like that shown in Parmer et al. U.S. Pat. No. 6,902,569, even if its ball-and-socket were placed substantially in the burr hole, still requires a hexagonal handled locking member (230) that protrudes substantially above the burr hole, which must be removed. However, such removal risks perturbing the accuracy of the carefully-placed instrument, even if it were to later be secured by some other means.

The present subject matter describes, among other things, a ball-and-socket trajectory guide in which the ball can be secured after trajectory alignment, such that the instrument can then be introduced through the ball along the aligned trajectory until it reaches the desired target, and such securing of the ball need not be later released during the procedure. Moreover, since the ball-and-socket and retaining member can be confined substantially within the burr hole, without protruding therefrom, the assembly can be left in place chronically for an ambulatory or other subject, such as by allowing skin to be sutured fully or partially closed above the assembly such as for improved appearance and decreased risk of infection, without creating the risk of bumping and injury from a substantial protrusion above the burr hole.

The present subject matter further describes a guide stem that can engage a ball passage to increase its effective bore length. In an example, the guide stem can be peeled apart or otherwise removed in sections with the instrument remaining in place, and such that the instrument can remain in place in the ball passage without being confined by the guide stem after the guide stem is removed. This can be particularly convenient if the instrument includes a proximal portion having a greater diameter than that of the instrument-guiding guide stem bore.

The present subject matter further describes a sealing cap with an injection port that can be used to cover the ball-and-socket in the burr hole, such as in a manner that can provide a fluid-retaining reservoir under the cap, which fluid can then be delivered to the target over an acute, extended, or chronic period of time, as desired.

The present subject matter further describes a first set of one or more user-visualizable or machine-visualizable, machine-imageable or other user-recognizable or machine-recognizable fiducial markers that can be provided and arranged to define a first plane that can be orthogonal to the trajectory. This can provide convenient image-guidance referencing, such as during alignment of the trajectory. The one or more fiducial markers defining the first plane orthogonal to the trajectory can define a first centroid on such first plane in a specified or determinable location. In an example, the one or more fiducial markers can be arranged such that the defined first centroid is at a location where the trajectory intersects the orthogonal first plane.

In a further example, a second set of one or more user-visualizable or machine-visualizable, machine-imageable or other user-recognizable or machine-recognizable fiducial markers can also be provided and arranged to define a second plane that can also be orthogonal to the trajectory, and spaced apart from the first plane. This can further provide convenient image-guidance referencing, such as during alignment of the trajectory. The one or more fiducial markers defining the second plane orthogonal to the trajectory can define a second centroid on such second plane in a specified or determinable location. In an example, the one or more fiducial markers can be arranged such that the defined second centroid is at a location where the trajectory intersects the orthogonal second plane (e.g., such as together with the first centroid being at a location where the trajectory intersects the orthogonal first plane).

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 shows an example of an exploded view of certain portions of a system that can include a trajectory guide apparatus and other components, which can be provided therewith.

FIG. 2 shows an example of another exploded view of certain portions of an example of the system including portions of the trajectory guide apparatus, where the removable guide stem has been removed from more distal access port components of the trajectory guide apparatus.

FIGS. 3A-3B show examples of respective cross-sectional views of an example of portions of the trajectory guide apparatus, in which the guide stem has been threaded into engagement with the ball passage of the ball.

FIGS. 4A-B show examples of respective cross-sectional views of an example of portions of the trajectory guide apparatus, in which the guide stem has been removed, an optional thread covering spacer, has been inserted, and a cap has been introduced.

FIG. 5 shows an exploded view of portions of an example of the trajectory guide apparatus in which an instrument can be disposed along the trajectory.

FIGS. 6A-B show cross-sectional views of an example of portions of the trajectory guide, respectively showing vertically and obliquely aligned instruments.

FIG. 7 shows a cross-sectional view of an example of portions of the trajectory guide, in which a rigid vertical cannula can be coupled with a flexible laterally-exiting catheter.

FIGS. 8 and 9A-9B respectively show an exploded view and an isometric view of an example of a platform or other structure that can be included in or used with the system and the trajectory guide, or with a different system or trajectory guide, or as an independent “alignment wand” without any other system or trajectory guide.

FIGS. 10A-10E show an example of using a single (e.g., ring-shaped) first fiducial marker arranged to define a first plane that is orthogonal to the trajectory, and a single (e.g., ring-shaped) second fiducial marker that can optionally be provided and arranged to define a second plane that is also orthogonal to the trajectory.

FIG. 11 shows an example of portion of the trajectory guide apparatus in which the retainer need not include threads.

FIG. 12 shows an example in which the ball can include an external protrusion, such as a threaded or other post, to which the guide stem can be user-engaged or user-mounted.

FIG. 13 shows an example that can include an optional infusion, drainage, or other port, such as can be located in or coupled to a cap.

FIG. 14 shows an example of such an alignment wand that can incorporate one or more of the features of the various fiducial marker arrangements described above, without requiring integration with a trajectory guide.

FIGS. 15A, 15B, and 15C show various views of an example of a base with a detent or restraint, such as a biocompatible clip.

FIGS. 16A (plan view) and 16B (cross-section view) show an example of a skull-mounted trajectory guide that can be mounted onto a subject's skull about a desired skull entry portal, such as a burr hole, such as for guiding an instrument through the skull entry portal and toward a desired path into the subject's brain.

FIG. 17 shows a thumb screw or guide pin portion of a skull-mounted trajectory guide.

FIG. 18 shows another view of the trajectory guide with a proximal portion of the guide stem shown.

FIGS. 19A, 19B, 19C, 19D, 19E, 19F, 19G, 19H, and 19I show an example of a guide stem that can include a “Z-Direction” height adjustment.

FIGS. 20A, 20B, 20C, 20D, and 20E show various views of an imaging fiducial stem that can be used together with the base.

FIGS. 21A, 21B, 21C, and 21D show an example in which the base of the trajectory guide can optionally include three or more legs, such as to permit the base to be raised above the burr hole or other entry portal.

FIG. 22 shows an example of a wrench or other tool that can be used to tighten the retainer ring to secure the ball in a desired position.

FIG. 23 shows an example of a skull mounted trajectory guide in which the base can be raised above the skull, e.g., along with the ball and the socket.

FIG. 24 shows an example of a “target-centered” skull mounted trajectory guide in which the base can be raised above the skull, such as described herein, such as described with respect to FIG. 23.

FIG. 25 shows an example of a “target-centered” skull mounted trajectory guide, such as described herein such as with respect to FIG. 24, but in which the arc can extend between two posts, such as to provide additional mounting stability for the arc.

FIG. 26 shows an example, similar to that shown and described above with respect to FIG. 20A, but in which a trajectory guide can include certain components having imageable fiducial markers.

FIGS. 27A-B shows an example, similar to that shown and described with respect to FIG. 23, in which the trajectory guide can include a base that can include an adjustable stage, such as for polar-offset or x-y adjustment.

FIGS. 28A, 28B, 28C, 28D, and 28E show an example of two or more concentric ring imageable fiducial marker rings, such as can be affixed to a proximal portion of the guide stem.

FIG. 29 is a diagram illustrating an example of trajectory guide alignment using a tapered arrangement of concentric rings.

### DETAILED DESCRIPTION

The present subject matter is described herein with particular emphasis toward an example in the general field of medicine relating to the introduction, placement, or stabilization of one or more devices in the brain, such as to treat a tumor or another neurological disorder. The present subject matter can help improve at least two key elements of this procedure: (1) precise and accurate instrument trajectory guidance, and (2) securing the delivered device, such as to permit short term use, long term use, or both.

In an example, the present subject matter can include a cranial access port or other trajectory guide apparatus or method, such as for the delivery and placement of a device into a human body, such as into the skull, in particular. The cranial access port device can be used with one or more other devices or instruments that can benefit from precise and accurate introducing, placing, and securing within the brain, such as, for an illustrative example, a catheter for infusing material into or draining material out of the body.

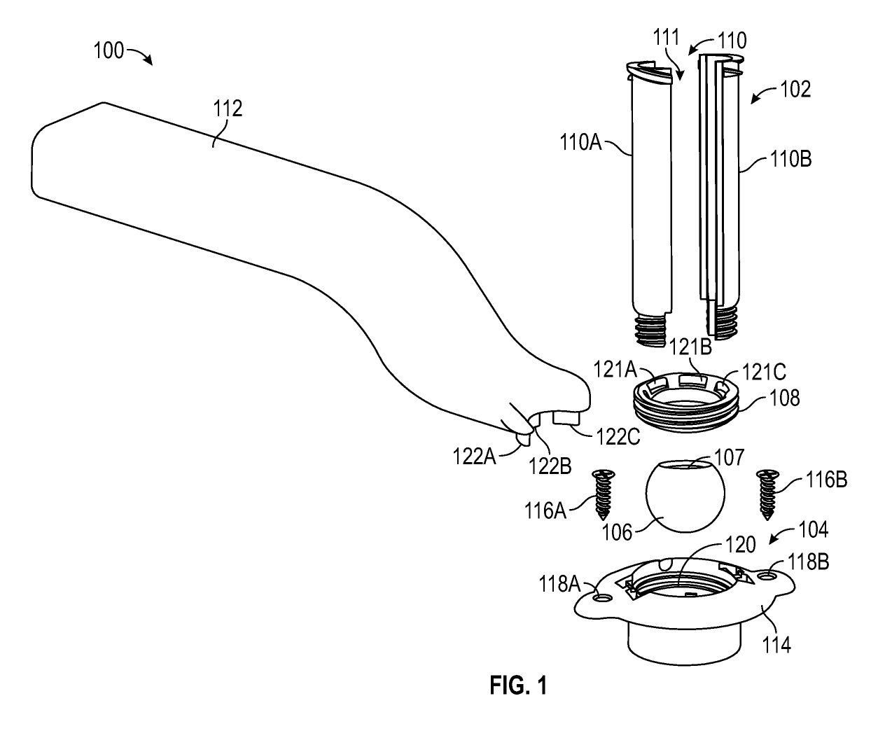

FIG. 1 shows an example of an exploded view of certain portions of a system 100 that can include a trajectory guide apparatus 102 and one or more other components, which can be provided therewith, such as in the form of a pre-packaged or other kit, such as within a sealed sterilized package, which can be accompanied by instructions for use (IFUs). In an example, the trajectory guide apparatus 102 can include a base 104, a spherical or other pivotable ball 106, and a ball-locking ring or other retainer 108. The combination of the base 104, the ball 106, and the retainer 108 can be sized, shaped, or otherwise configured to be located substantially within a burr hole that has been drilled into a subject's skull, without substantially protruding above such burr hole. (A typical dimension for a burr hole drilled into the subject's skull is 14 millimeters in diameter.) This can help allow chronic placement of the combination of the base 104, the ball 106, and the retainer 108, such as to allow overlying skin to be sutured partially or completely shut, which can help improve appearance or reduce the risk of infection, while avoiding or reducing the risk of bumping a protruding component, which could cause injury to the subject to which such a protruding component is mounted.

The trajectory guide apparatus 102 can also include a removable guide stem 110, such as can provide a bore 111 extending longitudinally between proximal and distal ends of the guide stem 110. The guide stem 110 can engage the ball 106, such as threadably, snap-in, or otherwise, such that the bore 111 of the guide stem 110 can be aligned concentric to a ball passage 107 that can extend through the ball 106, such as between proximal and distal ends of the ball 106. The ball passage 107, alone or in combination with the bore 111 of the guide stem 110, can establish a longitudinal trajectory concentric thereto, along which an instrument can be delivered to a desired target. The desired target can be located within the subject's skull. The desired target can be located at a shallow depth directly below the skull surface above the cortex (e.g., zero depth) or the desired target can be located in deep brain structures at the base of the skull (e.g., at depths of 15 cm-20 cm), or the desired target can be located anywhere therebetween. The guide stem 110 can protrude above the burr hole, but the guide stem 110 can be removed from more distal components of the trajectory guide 102. These more distal components of the trajectory guide 102 can be left in place without substantial protrusion above the burr hole. In an example, the guide stem 110 can be removed in peel-away, peel-apart, twist-apart, break-apart, or other sections 110A-B, as explained further below. In an example, the system 100 or kit can include a wrench or other tool 112, such as having one or more male or female or other engageable features configured for engaging one or more corresponding female or male or other engageable features of the retainer 108. The tool 112 can be used to secure the retainer 108 to the base 104, such as to lock the ball 106 in a desired pivoting position. This can hold a desired trajectory constant, such as aligned toward a desired target within the subject.

In an example, the base 104 can include or be coupled to an optional low-profile flange 114. The low-profile flange 114 can be sized, shaped, or otherwise configured to extend at least partially about the burr hole, including slightly above a plane defined by the surface of the burr hole. This can help to locate the base 104 substantially within the burr hole (e.g., except for the low-profile flange 114). In an example, the flange 114 (including any low-profile cap placed to cover over a receptacle defined by the flange 114) can provide a low enough profile, such as less than or equal to 5 millimeters above the outer surface of the skull, so as to be capable of allowing overlying skin to be sutured closed over the flange 114. This can help permit an access port portion of the trajectory guide 102, e.g., formed by a combination of the base 104, the flange 114, the ball 106 and the retainer 108 (after the guide stem 110 has been removed) to be left in place chronically, such as subcutaneously, and can help reduce or avoid the risk of injury from a protrusion being bumped, or can help improve appearance or reduce the risk of infection. The flange 114 can be secured to the skull, such as by one or more bone screws 116A-B or other fasteners or an adhesive. The bone screws 116A-B can be passed through respective holes 118A-B in the flange 114 and screwed into the skull, thereby securing the flange 114 and the base 104 in place.

The flange 114 is not required. In an example, the low-profile flange 114 can be omitted, such as in an example in which the base 104 is provided with outer circumferential threads that can permit the base 104 to be threaded into the bony structure of the skull, such as to secure the base 104 to the skull. The outer circumferential threads are also not required. Another fixation mechanism or technique can additionally or alternatively be provided to secure the base 104 to the skull, such as with or without the flange 114. In an example, an expansion element can be provided such as to expand an expandable portion of the base 104 to securely fit within the burr hole in the skull, such as using one or any of a number of expansion elements such as can be used in certain drywall mounts or like applications that can involve outward expansion.

The base 104 can include a socket 120 therein. The socket 120 can be sized, shaped, or otherwise configured to be located within (including beneath) the burr hole. The socket 120 can be sized, shaped, or otherwise configured to allow movement of the ball 106, such as pivoting. This can permit aiming the concentric trajectory of the ball passage 107 toward a desired target, such as within the subject's skull. The ball 106 can then be securely locked into place. This can hold the aimed trajectory constant, and can be accomplished such as by using the retainer 108. In an example, the retainer 108 can include one or more engageable features (e.g., male, female, or other) such as that do not protrude above a tangential plane defined by the top-most surface of the low-profile flange 114 or that of a similarly low-profile cap that can be placed over a receptacle provided by the base 104. This can help permit overlying skin to be sutured closed over the flange 114. As discussed above, this can help permit the access port portion of the trajectory guide 102, formed by the combination of the base 104, the optional flange 114, the ball 106, and the retainer 108, to be left in place chronically, such as subcutaneously, and can help reduce or avoid the risk of injury from a more substantial protrusion being bumped, as well as improving appearance or reducing the risk of infection. In the illustrative example of FIG. 1, such engageable features of the retainer 108 can include one or more receptacles 120. The receptacles 120 can be located or distributed about or near a circumferential periphery of the retainer 108. The receptacles 120 can be engaged by mating protrusions 122 on the tool 112, in an example. In this example, turning the retainer 108 using the tool 112 threads the retainer 108 onto the base 104. In this manner, the retainer 108 can be tightened down onto the base 104. This can lock the ball 106 in place to hold constant the trajectory provided by the ball-passage 107, such as after the trajectory has been aligned toward a desired target within the subject beyond the base 104. The secured ball 106 can later receive a diagnostic, therapeutic, or other instrument through the ball passage 107, such as for delivery or retention of the instrument along the locked aligned trajectory to the desired target.

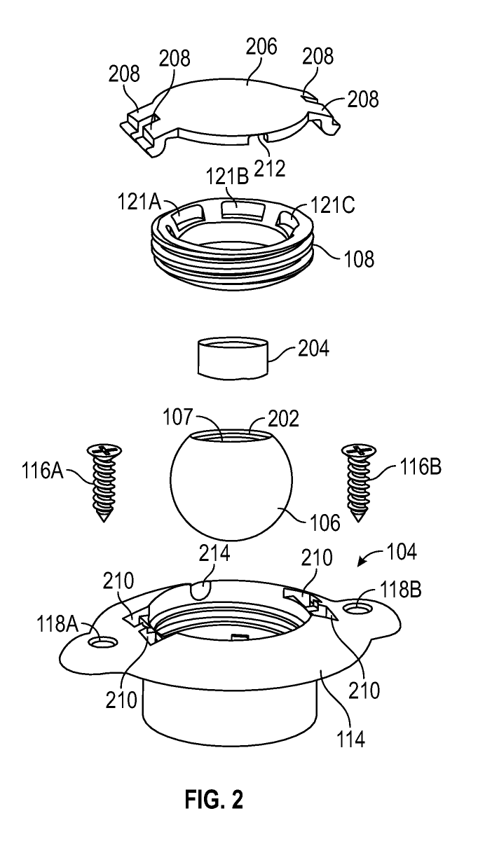

FIG. 2 shows an example of another exploded view of certain portions of the system 100 including portions of the trajectory guide apparatus 102. In the illustrative example of FIG. 2, the removable guide stem 110 has been removed from the more distal access port components of the trajectory guide apparatus 102. Such removal of the guide stem 110 can, in an example, include unthreading an outer thread, extending (e.g., externally) about a distal circumferential portion of the guide stem 110, from an inner thread 202, extending (e.g., internally) about a proximal circumferential portion of the ball passage 107.

After the guide stem 110 has been removed, it may be desirable to reduce or avoid risk of the inner thread 202 of the ball passage 107 chafing or otherwise damaging or affecting the instrument remaining at least partially within the ball passage 107. Therefore, after unthreading the guide stem 110 to remove it from the ball passage 107, a cylindrical or other thread cover spacer 204 can be inserted into the ball passage 107. The spacer 204 can be used to cover the thread 202 or to provide an interior through-lumen that can present a substantially smooth interior surface to a portion of the instrument that is located within the ball passage 107. In an example, the spacer 204 can provide a cylinder with a substantially smooth surfaced lumen therethrough. In an example, an outer surface of the cylindrical spacer 204 can include a thread. This can allow the spacer 204 to be threaded onto the inner thread 202 of the ball passage. In an example, the outer surface of the cylindrical spacer 204 can be ribbed. This can allow the spacer 204 to be pressed-fitted onto the inner thread 202 of the ball passage 107. In an example, the outer surface of the cylindrical spacer 204 can be smooth, such as where the cylindrical spacer 204 is to be held in place using a portion of the retainer 108. In an example, the cylindrical spacer 204 can be delivered to the ball passage 107 by introducing it over the instrument through the ball passage 107, such as by passing the instrument through the lumen of the spacer 204, and then passing the cylindrical spacer 204 over the instrument. In an example, a proximal edge portion of the lumen of the cylindrical spacer 204 can similarly taper outward so as not to present an abrupt edge to an instrument exiting the proximal end of the ball passage 107. The spacer 204 can be helpful to address potential chafing, etc., of the instrument by the present trajectory guide apparatus 102, but it can also be used in conjunction with any other ball-and-socket or other trajectory guide apparatus or other device in which an instrument can be left in place acutely or chronically against one or more threads in a passage through which the instrument passes.

The example of FIG. 2 also illustrates an example of a low-profile cap 206. The low-profile cap 206 can be placed substantially within the flange 114, in an example. This can include snap-fitting feet 208 (such as can extend laterally outward from the cap 206) into corresponding snap-fit feet-retaining shoes 210 that can be formed into the flange 114, or into the base 104 (such as where the flange 114 is omitted). The cap 206 can have a low profile, such as described above with respect to the low-profile flange, such that overlying skin can be sutured closed over the flange 114 and over the cap 206. As discussed above, this can help permit the access port formed by the combination of the base 104, the optional flange 114, the ball 106 and the retainer 108 to be left in place chronically, such as subcutaneously. This can help reduce or avoid the risk of injury from a substantial protrusion being bumped, such as in a chronic setting by an ambulatory subject. The cap 206 can have enough clearance below to allow the retainer 108 and the ball 106 to be operatively contained within the base 104 under the cap 206, which can be made of a clear material to allow visualization of these underlying components. In an example, the cap 206 can include or be made of a material that can seal against the flange 114 or the base 104. This can help retain fluid in a “reservoir” under the cap 206 and within the base 104. In an example the reservoir can include or be coupled to a micromechanical, electrokinetic, or other active or passive pump, such as for pumping or controlling delivery of the flowable substance to the desired target. For example, a passive pump can be included, such as to provide the reservoir, or coupled in fluid communication with the reservoir. The passive pump can include an at least partially elastic chamber, such as a bellows. The bellows can be filled with fluid, such as can cause the bellows to expand. As the bellows relaxes, it can provide a desired amount of positive pressure to urge infusate from the reservoir toward the target site.

In an example, the cap 206 can include an infusion or drainage or other port (e.g., a membrane, a valve, or the like). In an example, the port can be configured such that a flowable infusate can be injected or otherwise delivered through the port, such as using a syringe, an infusion pump, or other device. The port can be located at the center of the cap 206, or elsewhere. In an example, the cap 206 can include an instrument exit portal 214 to allow an instrument to exit (e.g., laterally) from under the cap 206. Such exiting can be either via the instrument exit portal 212 alone, or via the instrument exit portal 212 in the cap 206 in combination with an instrument exit portal 214 in the flange 114, which can be configured to align with the instrument exit portal 212 in the cap 206. In an example, one or both of these instrument exit portals 212, 214 can be configured to grip the instrument passed therethrough, such as to immobilize or stabilize it. In an example, one or both of these instrument exit portals 212, 214 can be configured to seal against the instrument exiting therethrough, such as to retain a flowable infusate under the cap 206.

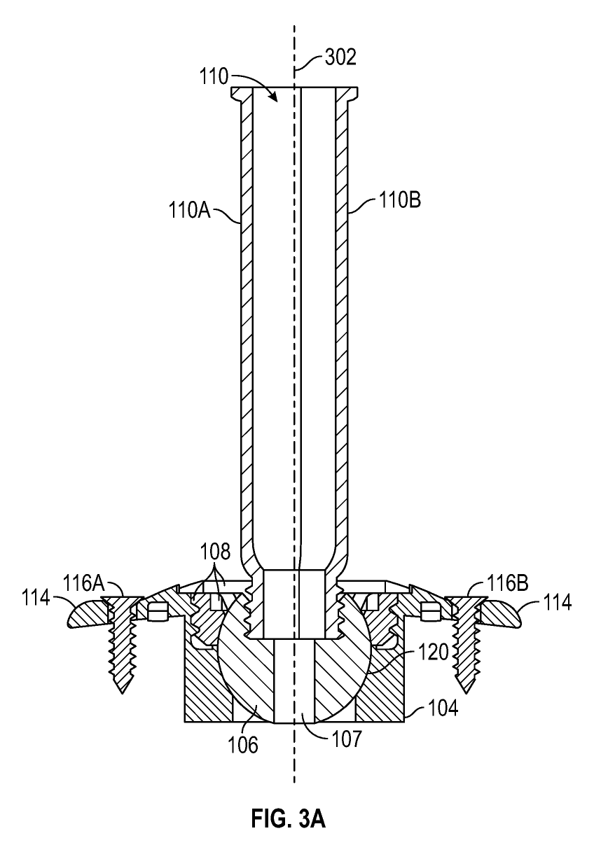

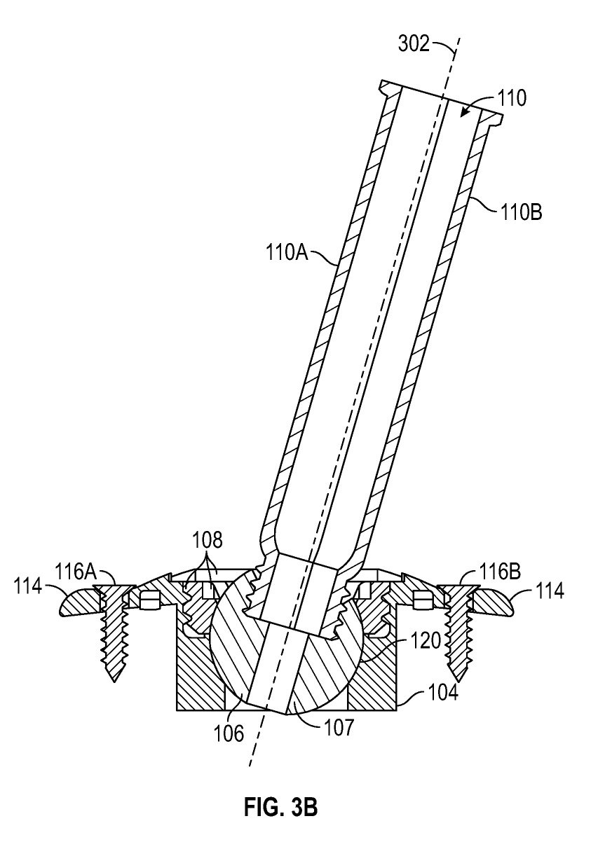

FIGS. 3A-B show examples of respective cross-sectional views of an example of portions of the trajectory guide apparatus 102, in which the guide stem 110 has been threaded into engagement with the ball passage 107 of the ball 106. The base 104 can be sized, shaped, or otherwise configured to include a socket 120 portion into which the ball 106 is seated, such as with a central pivot point of the ball 106 located below the surface of the skull. The socket 120 can be shaped to allow smooth pivoting of the ball 106 with respect to the socket 120. This can permit pivoting the ball 106, such as to obtain a desired alignment of the trajectory 302 toward a desired target within the skull. The retainer 108 can be threaded into the base 104, such as to securely lock the ball 106 down against the socket 120 portion of the base 104, such as to hold constant a desired trajectory 302. FIG. 3A shows an example in which the guide stem 110 can be vertically aligned with respect to the base 104, e.g., substantially parallel to an axis extending concentrically through the burr hole orthogonal to the skull. The retainer 108 can lock the ball 106 into this orientation. FIG. 3B shows an example in which the ball 106 can be pivoted with respect to the socket 120, such that the guide stem 110 can be aligned at an angle with respect to the axis extending concentrically through the burr hole orthogonal to the skull. The retainer 108 can lock the ball 106 into this orientation.

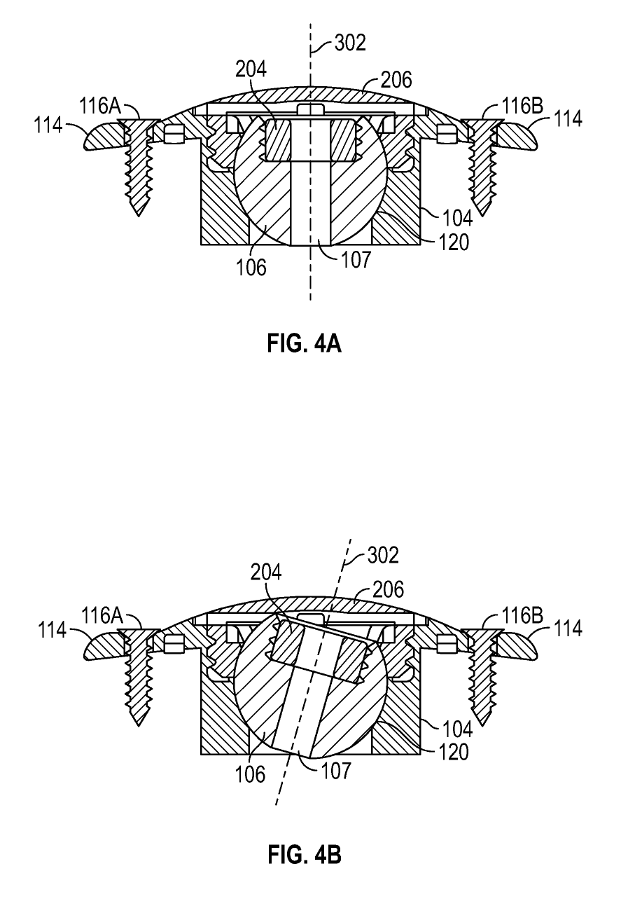

FIGS. 4A-B show examples of respective cross-sectional views of an example of portions of the trajectory guide apparatus 102, in which the guide stem 110 has been removed from engagement with the ball passage 107 of the ball 106. An optional thread covering spacer 204 has been inserted into a proximal portion of the ball passage 107 of the ball 106. A cap 207 has been snap-fitted or otherwise engaged into the flange 114 or the base 104. FIG. 4A shows an example in which the guide stem 110 can be vertically aligned with respect to the base 104, e.g., substantially parallel to an axis extending concentrically through the burr hole orthogonal to the skull. The retainer 108 can lock the ball 106 into this orientation. FIG. 4B shows an example in which the ball 106 can be pivoted with respect to the socket 120, such that the guide stem 110 can be aligned at an angle with respect to the axis extending concentrically through the burr hole orthogonal to the skull. The retainer 108 can lock the ball 106 into this orientation.

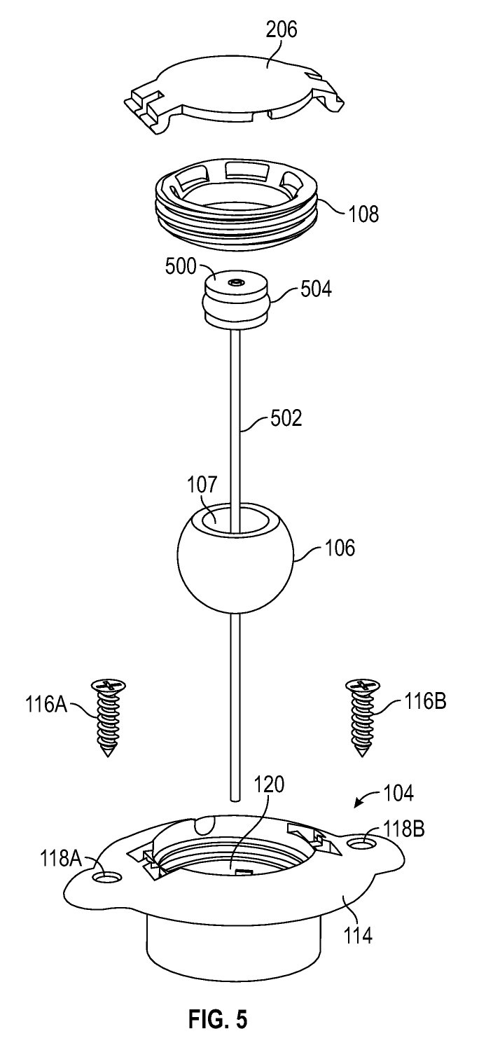

FIG. 5 shows an exploded view of portions of an example of the trajectory guide apparatus 102 in which the instrument can include a rigid or other cannula 502, such as can be disposed along the trajectory 302. A distal end of the cannula 502 can be positioned at or near a desired shallow, deep, or other target within the skull. A proximal end of the cannula 502 can be received in a hub 500, such as within a central lumen of the hub 500. In an example, the hub 500 can seal against or otherwise engage a portion of the ball 106, such as within the ball passage 107. In an example, the hub 500 can seal against or otherwise frictionally engage an inner circumference of the ball passage 107. This can inhibit longitudinal movement of the hub 500 within the ball passage 107. Additionally or alternatively, this can seal against, or prevent fluid flow between the hub 500 and the inner circumference of the ball passage 107. In an example, the hub 500 can include or be coupled to an O-ring 504 or other seal extending about an outer circumference of the hub 500, such as to provide the sealing or other frictional engagement described above. In an example, the hub 500 can include a compliant stopper-like component, such that no separate O-ring 504 or seal is needed to provide the sealing or other frictional engagement. In an example, the cannula 502 itself can directly seal against or otherwise engage the inner circumference of the ball passage 107, such as where sealing or other frictional engagement is not needed, for example, if a nail-head-like or other stop of the cannula 502 comes to rest upon a proximal end of the ball 106. In an example, the cap 206, the optional flange 114, and portions within the base above the hub 500 can form a reservoir, such as for storing a flowable substance that can then be delivered to the desired target location, such as via the cannula 502.

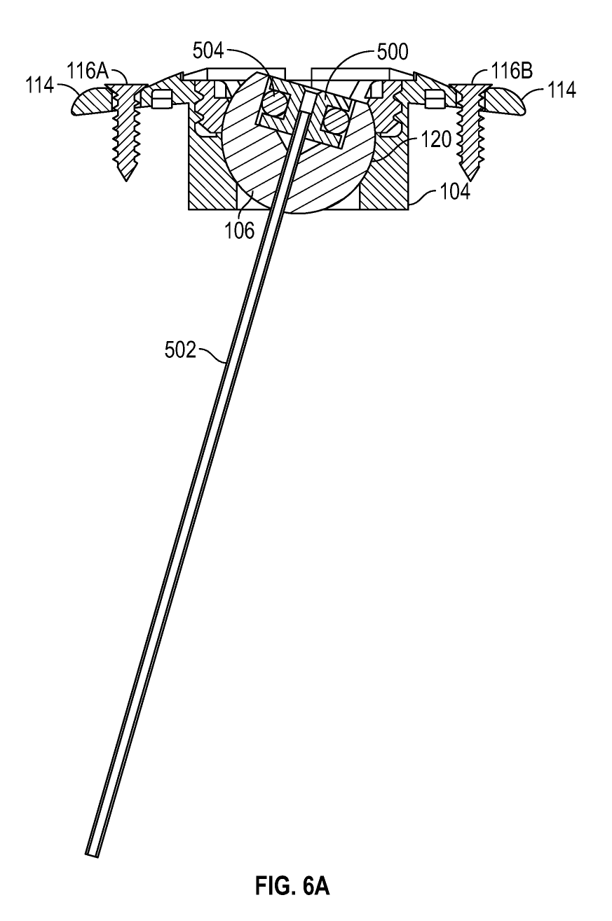

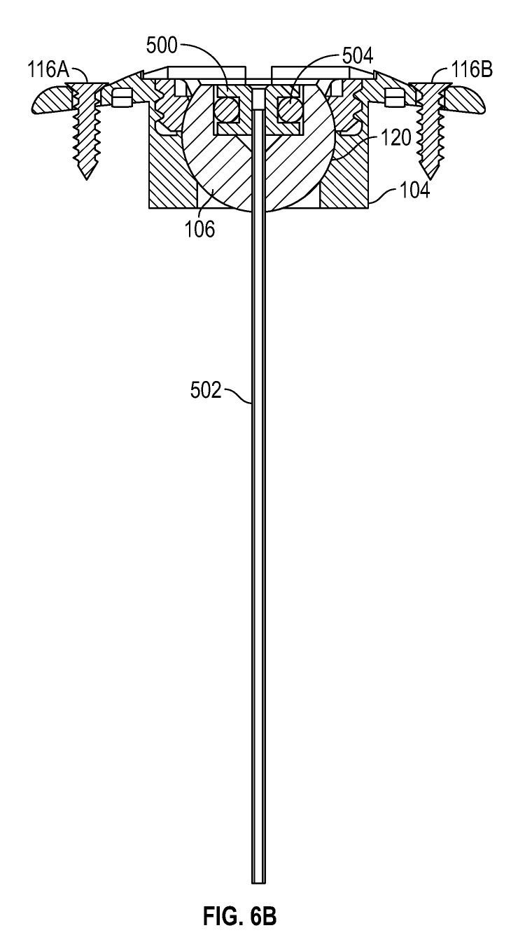

FIGS. 6A-B show cross-sectional views of an example of portions of the trajectory guide 102, respectively showing vertically and obliquely aligned cannulas 502, extending distally into the subject's skull from a proximal end of the cannula 502 that is engaged within the ball passage 107 of a ball 106 in a socket 120 of a base 104, such as sealingly or frictionally via the hub 500 or a component associated therewith, such as the O-ring 504 or other seal or brake.

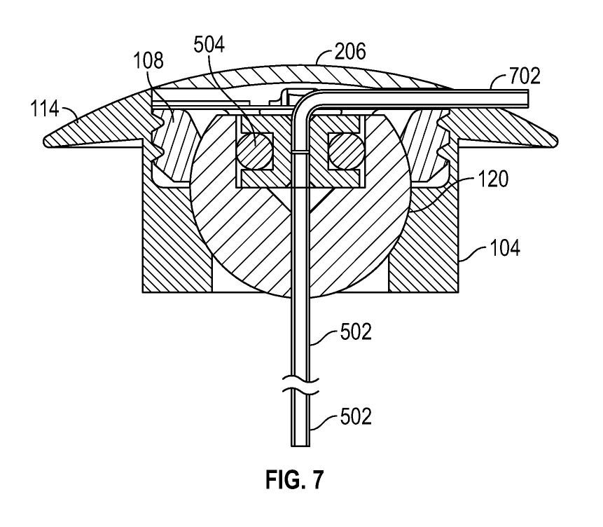

FIG. 7 shows a cross-sectional view of an example of portions of the trajectory guide 102, in which the rigid vertical cannula 502 can be coupled (e.g., in fluid communication) with a flexible laterally-exiting catheter 702. In this way, the lateral catheter 702 can be easily connected to or disconnected from the cannula 502, such as without disturbing the intracranial cannula 502 or the access port portion of the trajectory guide 102 to which the intracranial cannula 502 is secured. In an example, the lateral catheter 702 can help facilitate both connection to or removal from the intracranial cannula 502, such as at some time after the procedure in which the access port components of the trajectory guide 102 have been installed onto the subject. For example, it may be desirable to allow the associated installation incision to heal for some period of time before connecting the lateral extension line provided by the lateral catheter 702, or it may be desirable to “yank” out or otherwise remove the lateral extension catheter 702 without disturbing the intracranial cannula 502.

FIG. 11 shows an example of portion of the trajectory guide apparatus 102 in which the retainer 108 need not include threads. In an example, the retainer 108 can include a retainer ring 1102 that can be seated upon the ball 106 such as to secure the ball 106 in place such as with the ball passage aligned to maintain a desired trajectory. In an example, the retainer ring 1102 can include a pinned or other hinge 1104 that can couple the retainer ring 1102 directly or indirectly to the base 104. The hinge 1104 can be located on one side of the retainer ring 1102, such that the retainer ring 1102 and the hinge 1104 can be configured in a manner similar to that of a hinged toilet seat. The other side of the retainer ring 1102 can include a fixation mechanism 1106 that can be configured to secure the retainer ring 1102, such as to press firmly against the ball 106 to hold the ball 106 in place and to inhibit pivoting by the ball 106. The fixation mechanism 1106 can include a male or female or other snap-fitting feature such as can be user-engaged directly or indirectly to the base 104, or can include a screw or any other fastener. The retainer ring 1102 can be configured such that when the fixation mechanism 1106 is engaged to the base 104, the retainer ring 1102 can be located substantially within the burr hole, such as to allow optional placement of the overlying cap 206, and such as to allow overlying skin to be sutured partially or fully closed, as desired, such as described above.

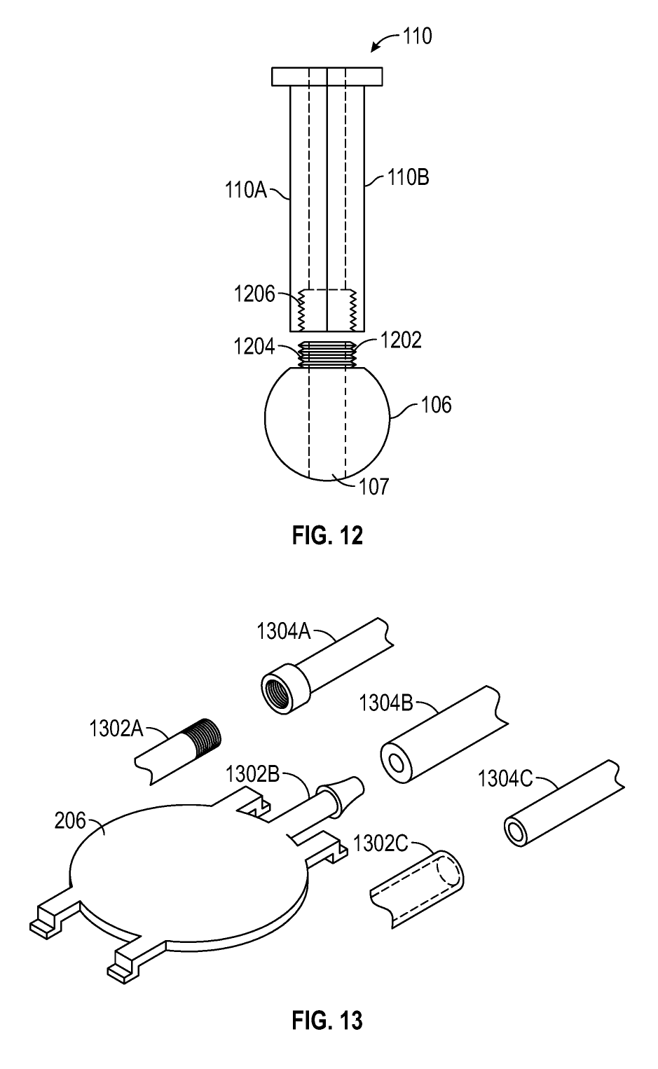

FIG. 12 shows an example in which the ball 106 can include an external protrusion, such as a threaded or other post 1202, to which the guide stem 110 can be user-engaged or user-mounted. In an example, the post 1202 can include one or more threads, such as external threads 1204 extending about the circumferential periphery of the post 1202 to which corresponding internal threads 1206 or one or more other engageable features within an internal distal portion of the guide stem 110 can be threaded or otherwise engaged. The example shown in FIG. 12 can avoid needing internal threads within the ball passage 107 which, as explained above, can chafe against an instrument passing through the ball passage 107. This, in turn, can avoid any need for the optional cylindrical spacer 204 described above. The protrusion need not include a post 1202, but can include a ring, a lip, or other external feature of the ball 106. Engagement of the guide stem 110 to such post 1202 or other protrusion need not include threading, but can instead be via snap-fitting or another engagement mechanism or technique than can be performed by the user.

FIG. 13 shows an example that can include an infusion, drainage, or other port 1302, such as can be located in or coupled to the cap 206. In an example, the port 1302 can exit the cap 206 laterally, such as to help permit overlying skin to be sutured partially or preferably completely closed, such as explained above. The port 1302 can include one or more features to allow it to be frictionally coupled to or otherwise engaged to or sealed against a catheter 1304 or the like, which can be coupled to the port 1302, such as to allow infusion or drainage. In an example, a port 1302A can include external or internal threads that can be configured to threadably engage corresponding internal or external threads of an end portion of the catheter 1304A. In an example a port 1302B can include a lip that can be configured to retain a compliant end portion of the catheter 1304B. In an example, a portion 1302C can include a female receptacle into which a distal end portion of the catheter 1304C can be inserted. Thus, the port 1302 can be configured to inhibit inadvertent pulling of the catheter 1304 away therefrom.

Portions of the system 100 or the trajectory guide apparatus 102 can be constructed of suitable biocompatible or MRI compatible materials. To recap, the system 100 and trajectory guide apparatus 102 can provide trajectory guidance for an diagnostic, therapeutic, or other interventional instrument, such as based on the user's knowledge of the subject's anatomy, which may be based on real-time or pre-operative magnetic resonance (MR), computed tomography (CT), or another imaging modality. Advantages of the present systems, devices, or methods can include, among other things, that the trajectory guide 102 can include cranial access port components—including the ball 106 and the retainer 108—which need not be removed, or even unsecured after the trajectory is aligned to prepare for introducing the instrument through the trajectory guide 102. This can help provide more accurate delivery of the instrument to the desired target. Moreover, the access port components of the trajectory guide apparatus 102 can be located substantially within the burr hole or can provide a low-profile so as to allow the skin to be sutured closed over such access port components of the trajectory guide apparatus 102. The guide stem 110 can be removed while leaving the instrument in place through the ball passage 107—even if the instrument includes a proximal bulge that is wider than the bore 111 of the guide stem 110 because, in an example, the guide stem 110 can be split into two or more sections, such as the sections 110A, 110B. Furthermore, it can maintain a cannula 502 or other conduit to the target, while providing for attachment thereto, such as by a flexible lateral catheter 702, which can be tunneled beneath the scalp. The cannula 502 can be used to deliver a substance to the target or to drain a substance from the target. Alternatively or additionally to the tunneled lateral catheter 702, the cap 206 can include a port, such as an injection port, such as to deliver a flowable substance (e.g., a drug or other infusate) to the target, such as through the cannula 502.

### Fiducial Marker Arrangement Examples

Another aspect of the present systems, devices, and methods can include subject matter related to medical imaging, and more particularly, to localizing an interventional (e.g., diagnostic, therapeutic, or other) device within or around the body of a human or animal subject, such as by using a medical imaging system (e.g., MRI, fMRI, CT, PET, ultrasound, or another imaging modality).

The system 100 and trajectory guide 102 described above (as well as other trajectory guide devices that need not have the features or advantages of the trajectory guide 102 described above) can be used for delivering an interventional instrument to a desired area of the human or animal body, such as a desired target within the human or animal body. This can be done under guidance of a pre-operative or real-time imaging modality. For example, a typical MRI is capable of clearly imaging soft tissue and fluid within the bore of the MRI magnet.

One approach can be to visually align a trajectory, such as by using a tube filled with an imaging-recognizable fluid, such as saline or a combination of saline and gadolinium, which can enhance the imaging visibility of the fluid. In this approach, the tube can then be held concentrically within a bore of a guide stem of a trajectory guide, and the trajectory guide can then be visually aligned with the desired target. In this approach, the fluid-filled tube can then be removed and replaced with the instrument being delivered. The present inventors have recognized that in this approach, however, after the fluid-filled tube is removed from the guide stem of the trajectory guide, alignment can no longer be confirmed using the imaging modality.

Accordingly, the present inventors have recognized that another approach can be provided. At least three points or other objects can be used as user-visualizable or machine-imageable or other fiducial markers, such as to define a plane. If those points also define a geometric shape on that plane, such as, for example, a triangle (e.g., using at least 3 points), a rectangle (e.g., using at least 4 points), a pentagon (e.g., using at least 5 points), etc., such a centroid (a unique center of area) may be defined on that plane with respect to those fiducial marker points and the shape that they create. If such fiducial marker points are visible in an MRI or other imaging modality's image, they can define a unique axis that is (a) perpendicular to the plane and (b) intersecting the centroid. If such fiducial markers are arranged on the defined plane such that the fiducial markers themselves do not lie along such trajectory axis defined perpendicular to their shared common centroid, then such fiducial marker points can be left in place to allow imaging during instrument delivery without obstructing the trajectory path. This can allow for the delivery of an instrument while concurrently providing alignment confirmation using the imaging modality, whether MR, CT, or any other imaging modality in which the fiducial marker points can be made visible. Moreover, a “depth to target” can be measured, such as the distance from such centroid (or referenced thereto, such as from a proximal end of the guide stem 110) to the target along the trajectory axis. Furthermore, a second point along the trajectory axis can be defined as a virtual “probe-tip” or “pointer,” such as to allow for localization of one or more points of interest within the image.

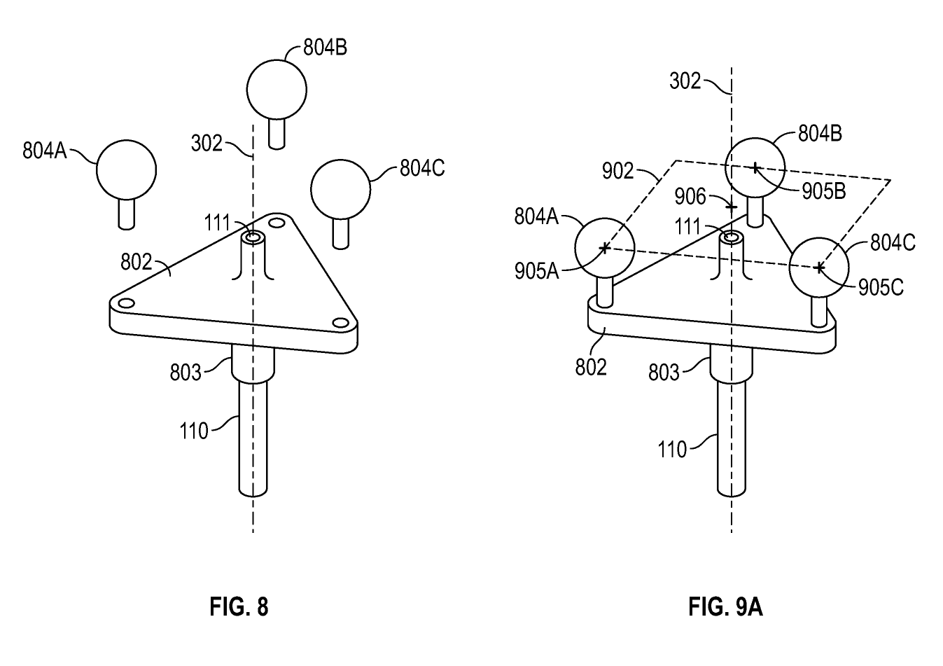

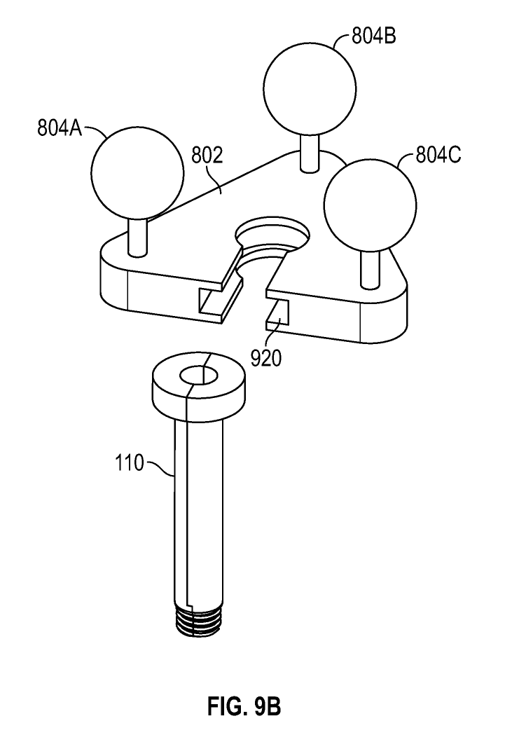

FIGS. 8-9A-B respectively show an exploded view and an isometric view of an example of a platform 802 or other structure that can be included in or used with the system 100 and the trajectory guide 102 described above (or with one or more other instruments or trajectory guide devices that need not have the features or advantages of the trajectory guide 102 described above). The platform 802 can be used to help performing imaging, such as for locating a desired area on a human or animal subject, such as for delivering an interventional instrument to a desired area of the human or animal body, such as to a desired target within the human or animal body. In an example, the platform 802 can be secured, affixed, or mounted to the guide stem 110 (or to an instrument to be passed through the bore 111 of the guide stem 110). The guide stem 110 can have two user-separable parts 110A-B, such as described above. The platform 802 can be secured such that the platform 802 does not obstruct the bore 111 of the guide stem 110, such as by allowing the bore 111 of the guide stem 110 to pass through the platform 802 in an example, or by allowing the bore 111 of the guide stem 110 to coaxially align with a corresponding bore portion of the platform 802 that effectively extends the bore 111 of the guide stem 110. For example, FIG. 9B shows an example in which a proximal portion of the guide stem 110 can be slid into a slot 920 in a side of the platform 802 such that the platform 802 can be centered upon the guide stem 110. This can effectively allow the bore 111 of the guide stem 110 to pass through the platform 802 or at least be unobstructed by the platform 802. This can permit the instrument 111 to still be passed through the bore 111 of the guide stem 110. In an example, the platform 802 can be affixed to the guide stem 110 by a hollow center post 803 portion of the platform 802. The post 803 can extend orthogonally from the platform 802 such that the post 803 can slip snugly over the guide stem 110.

An arrangement of one or more machine-imageable fiducial markers 804 can be located on the platform 802. The fiducial markers 804 can be placed at specified locations on the platform 802 such as to define a first plane 902 that is orthogonal to the longitudinal trajectory 302 defined concentrically to the bore 111 of the guide stem 110. In the example of FIG. 8, this can include three substantially spherical (or other centroid-defining) MRI-visible fluid-filled fiducial markers 804A-C, which can each define a respective concentric center 905A-C of the corresponding individual spherical fiducial marker 804A-C. The individual centroids 905A-C of the respective fiducial markers 804A-C can collectively define a specified or determinable common centroid 906 on the first plane 902, wherein the first plane 902 is orthogonal to the trajectory 302. In an example, the MR or other imaging modality being used can recognize the locations of the centroids 905A-C of the fiducial markers 804A-C, such as by using an image-processing circuit that coupled to the MR scanner. From these centroid locations 905A-C, the image-processing circuit can determine the orientation of their common first plane 902, and can compute the location of the common centroid 906 within the common first plane 902. The common centroid 906 within the common first plane 902 can be located at its intersection with the orthogonal trajectory 302, or in a specified relationship thereto, such as by appropriate selection of the physical locations of the fiducial markers 804A-C on the platform 802.

The platform 802 can include threaded receptacles into which respective posts extending from the spherical portions of the fiducial markers 804A-C have been threaded. Each post can be precisely configured with a threaded distal portion to be inserted to a specified depth. Accordingly, when the posts of the fiducial markers 804A-C are inserted into the platform 802, the first plane 902 defined by the respective centroids 905A-C of the fiducial markers 804A-C is orthogonal to the trajectory 302 through the bore 111 of the guide stem 110.

In the example of FIGS. 8-9, the common centroid 906, defined by the individual centroids 905A-C of the respective fiducial markers 804A-C, is located at the intersection between the first plane 902 and the orthogonal trajectory 302 extending through the bore 111 of the guide stem 110 over which the hollow post 803 has been fitted. However, in other examples, the common centroid 906, defined by the individual centroids 905A-C of the respective fiducial markers 804A-C, can be located in another (different) specified or determinable (e.g., by the image-processing circuit) location with respect to at the intersection between the first plane 902 and the orthogonal trajectory 302 extending through the bore 111 of the guide stem 110 over which the hollow post 803 has been fitted.

In a real-time MR imaging example, the platform 802 and the guide stem 110 or other components of the system 100 can be made of any suitable non-magnetic material, such as plastic, ceramic, carbon fiber, or the like. In a CT example, a metal or metal alloy, such as aluminum or stainless steel can be used, in addition or as an alternative to plastic, ceramic, carbon fiber or the like. In an example, the platform 802 can be integrally constructed as part of the guide stem 110. In an MR imaging example, the fluid-filled fiducial markers 804 can include a container material that can be made of plastic, glass, ceramic, or carbon fiber, which can be filled with an imageable fluid such as saline, gadolinium, a mix, or any medium that is visible in the imaging modality used.

While the example described above with respect to FIGS. 8-9 has particularly emphasized a specific device and method of aligning a trajectory 302 for introducing an instrument using MRI, these techniques can also be applied to CT or another imaging modality in which their locations can be made visible. Unlike an approach in which an imager-visible fluid-filled stem is inserted into the bore 111 of the guide stem 110, and then removed for allowing subsequent instrument insertion, the platform 802 and the fiducial markers 802A-C can be left in place during insertion of the cannula 502 or another instrument into the bore 111 of the guide stem 110, thereby allowing real-time imaging verification of the alignment even during instrument delivery.

While the example described above with respect to FIGS. 8-9 has particularly emphasized and illustrated the use of multiple (e.g., three) separate discrete fiducial markers 804A-C, other arrangements one or more fiducial markers can additionally or alternatively be used to define the first plane 902. For example, a single contiguous substantially flat ring-shaped fiducial marker can also be used to define the first plane 902, and can define a centroid within the first plane 902 that is at a specified or determinable location with respect to the orthogonal trajectory 302. Similarly, one or more other two dimensional (2D) or three dimensional (3D) shapes can be used to define a plane and a specified or determinable centroid in that plane—and can leave unobstructed the bore 111 of the guide stem 110, thereby allowing real-time imaging verification of the alignment of the trajectory 302, even during instrument delivery.

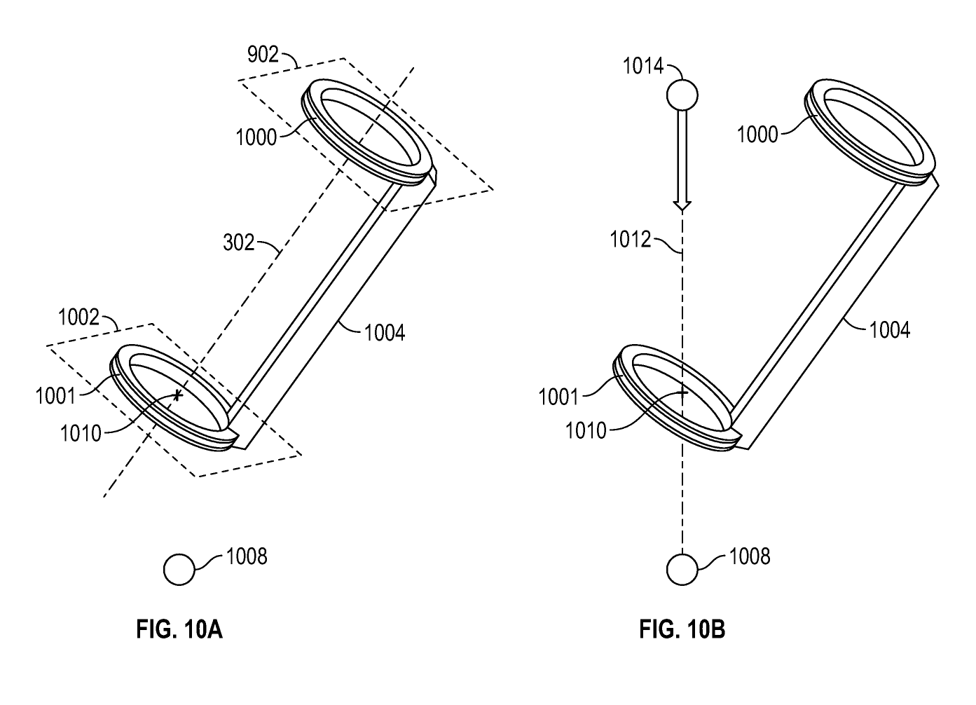

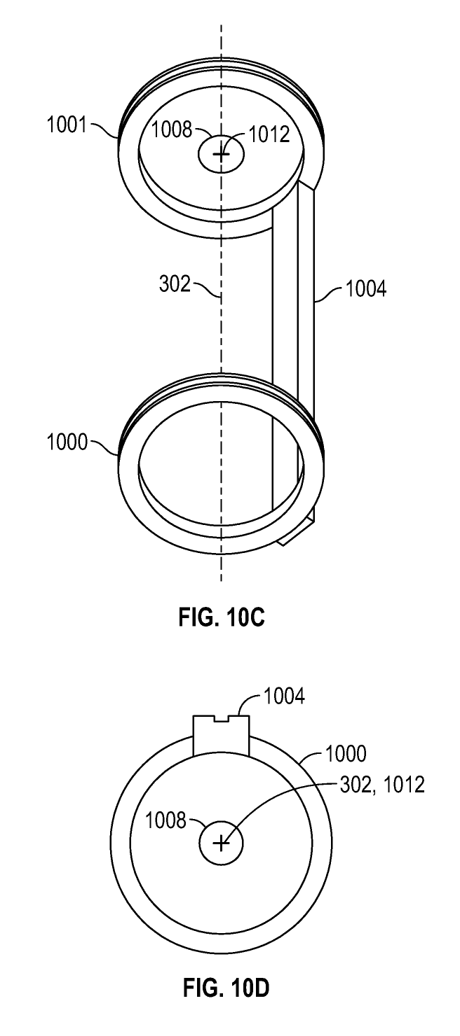

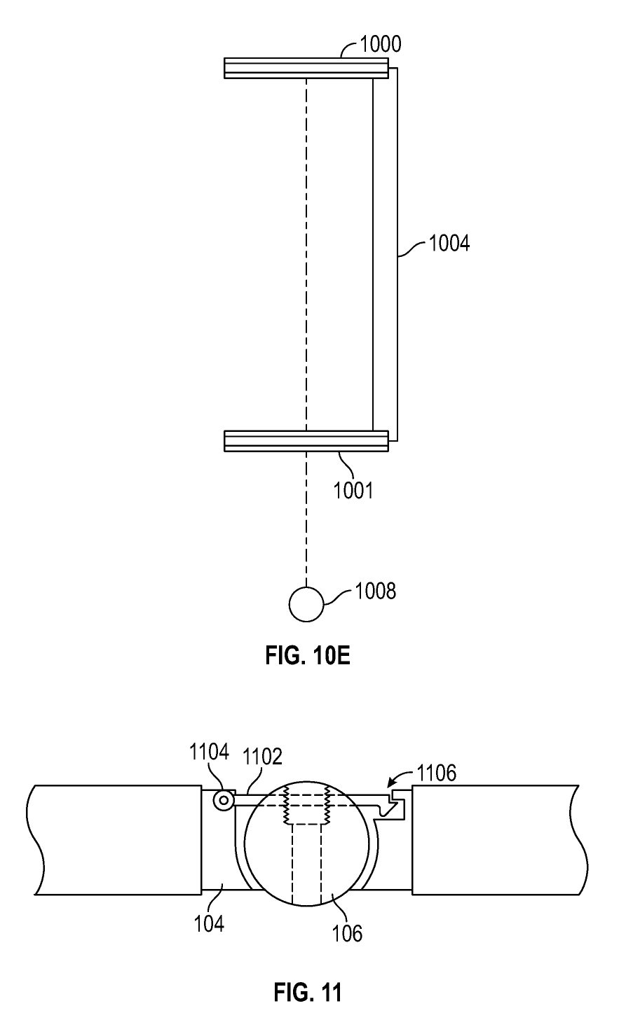

FIGS. 10A-10E show an example of using a single (e.g., ring-shaped) first fiducial marker 1000 arranged to define a first plane 902 that is orthogonal to the trajectory 302. A single (e.g., ring-shaped) second fiducial marker 1001 can be provided and arranged to define a second plane 1002 that is also orthogonal to the trajectory 302. The second plane 1002 is spaced-apart from the first plane 902 by a specified distance. The first plane 902 can be spaced-apart from the second plane 1002 by mounting, securing, or otherwise affixing the first and second fiducial markers 1000, 1001 to a connector beam 1004, which can serve as a tie or strut between the fiducial markers 1000, 1001. In an example, the guide stem 110 itself can serve as the connector beam 1004. In an example, the instrument to be inserted to the target 1008 via the bore 111 of the guide stem 110 along the trajectory 302 can serve as the connector column 1004.

In the example of FIGS. 10A-E, the ring fiducial markers 1000, 1001 can define respective centroids 1010A-B in the first plane 902 and the second plane 1002, respectively, such as at the respective intersections of these planes with the trajectory 302 passing orthogonally through each, or in a (preferably like, but possibly different) specified or determinable distance and relationship to the trajectory 302.

FIGS. 10A-10E show an example in which the ring fiducial markers 1000, 1001 can each be arranged to be concentric to the trajectory 302, each providing a respective centroid located at an intersection of the trajectory 302 through the respective planes defined by the rings of the fiducial markers 1000, 1001. In the example of FIG. 10A, the rings can have like diameters. In another example, however, the rings can have different diameters, for example, such that at least a portion of each is concurrently visible when looking directly down the trajectory 302.

FIG. 10A illustrates an example in which the fiducial markers 1000, 1001 can be arranged with respect to the trajectory guide apparatus 102 so as to pivot about a pivot point 1010 in the second plane 1002, such as a pivot point 1010 defined by the centroid of the fiducial marker 1001. The trajectory 302 passes through the pivot point 1010 orthogonal to the second plane 1002.

FIG. 10B illustrates an example of how, for example, using the imaging modality, a desired line 1012 can be constructed through a centroid of the target 1008 and through the pivot point 1010 defined by the centroid of the fiducial marker 1001. Using the imaging modality, in an example, a point 1014 can be selected along the desired line 1012 and above the ring fiducial marker 1000. In an example, selection of the point 1014 can be subjective, such as based on a visual estimation of the centroid of the target 1008, either using the imaging modality or using visualization without the imaging modality.

FIG. 10C illustrates an example of how, using the imaging modality to view axially down the desired line 1012, the ball 106 can be pivoted in the socket 120 such that the trajectory 302 aligns with the desired line 1012, thereby pointing the trajectory 302 toward the target 1008. In FIG. 10C, the desired line 1012 is perpendicular to the page of the drawing, and is not yet aligned to the trajectory 302.

FIG. 10D shows how such alignment between the trajectory 302 and the desired line 1012 can be accomplished by aligning the ring fiducial markers 1000, 1001 until they visually align concentrically, such as when viewed with the imaging modality down along the desired line 1012. This can be conceptualized as sighting a target through two ring-shaped gun sights. The trajectory 302 is then aligned to the target 1008. This is shown in FIG. 10D, with both the desired line 1012 and the trajectory 302 being perpendicular to the page of the drawing. The ball 106 can then be secured, such as by using the retainer 108, as described above. The cannula 502 or another instrument can then be introduced along the trajectory 302 to the target 1008. Such alignment can be accomplished using the imaging modality, visually, or a combination thereof. Although FIG. 10D shows an example with like-diameter ring fiducial markers 1000, 1001, different-diameter ring fiducial markers 1000, 1001 can also be used.

FIG. 10E shows how a depth-to-target measurement may be made, using the imaging modality, in an orthogonal view to the trajectory 302. One or more of the rings of the fiducial markers 1000, 1001 can be arranged in a specified or determinable spatial relationship to a reference point at which the instrument is to be inserted. In this way, such a ring can be used as a reference for the depth-to-target measurement, which can be made in the orthogonal view using the imaging modality.

With respect to the examples shown in FIGS. 10A-10E, in other examples, there can be more than two ring or other fiducial markers 1000, 1001 provided, such as to provide additional corresponding planes that are orthogonal to the trajectory 302. Moreover, the fiducial markers 1000, 1001 need not have ring shapes—other symmetrical shapes or other shapes, from which a centroid can be determined (e.g., without the shape blocking the bore 111 of the instrument guide 110), can also be used. Such shapes need not be a single continuous shape, like a ring. For example, multiple discrete shapes, such as the spherical fiducial markers 804A-C described above, can also be used to define a plane and a common centroid within that plane. In an example, the fiducial markers 1000, 1001 can be auto-detected by software, such as can be performed on the image-processing circuit that is included in or coupled to the imaging modality. Enhanced visibility using the imaging modality can be facilitated using fluid, metal, or other MR or radio-opaque materials, as appropriate for the particular imaging modality selected.

The above description of the various fiducial marker structures has emphasized how they can be included in or used with the systems and the trajectory guides described herein. However, such fiducial marker structures and methods can also be used with other systems or trajectory guides, or even without an accompanying trajectory guide, such as an independent “alignment wand” that can be used with an imaging modality or other machine-assisted visualization system.

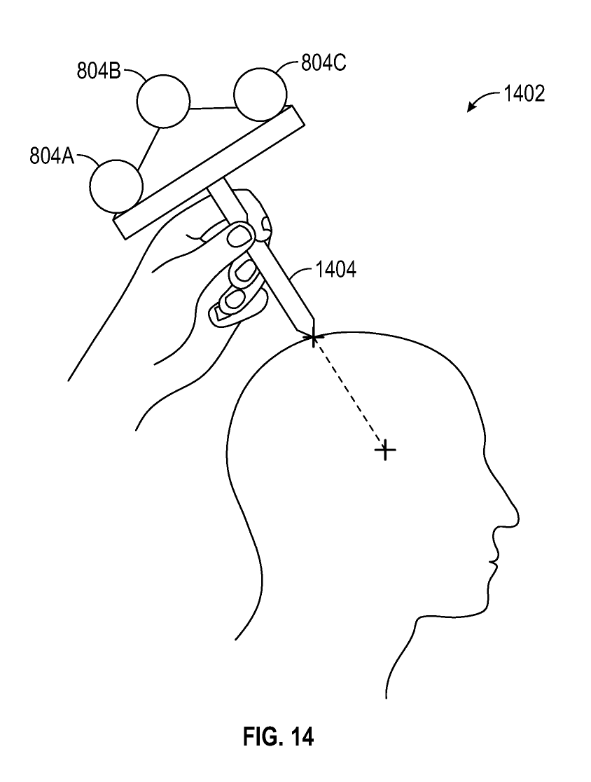

FIG. 14 shows an example of such an alignment wand 1402 that can incorporate one or more of the features of the various fiducial marker arrangements described above, without requiring integration with a trajectory guide, but allowing for use with or without such a trajectory guide, as desired. In an example, the alignment wand 1402 can include a post 1404, which can optionally be configured to be inserted smoothly and snugly into the bore 111 of the guide stem 110. Regardless of whether it is inserted into the guide stem 110, the post 1404 can define a planned instrument trajectory 302, and can include an arrangement of fiducial markers 804A-C arranged on a platform 802 to define a plane orthogonal to the trajectory 302, such as described above, for example, with respect to FIG. 8.

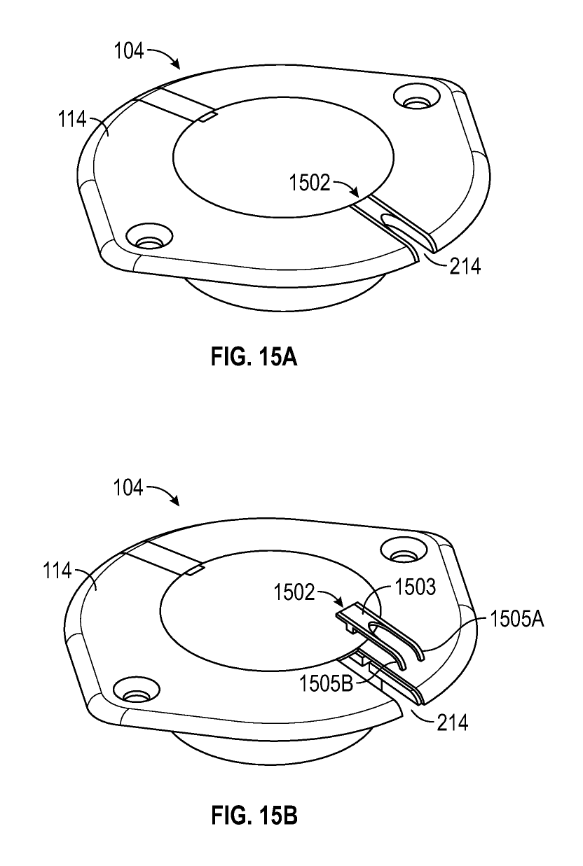

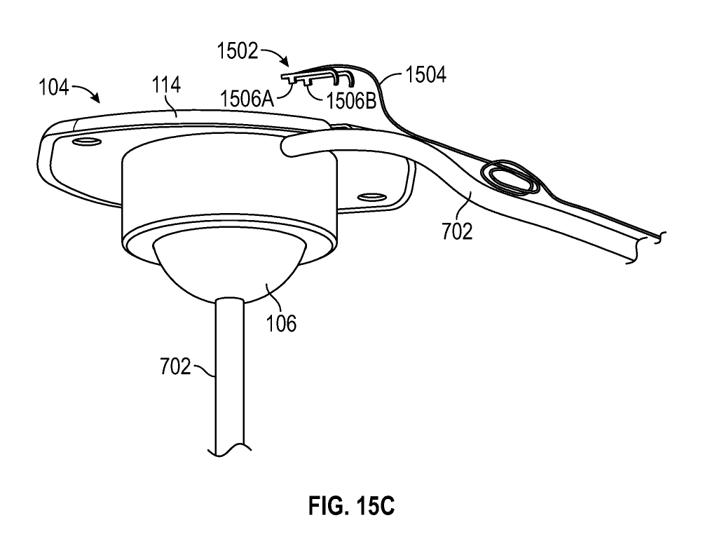

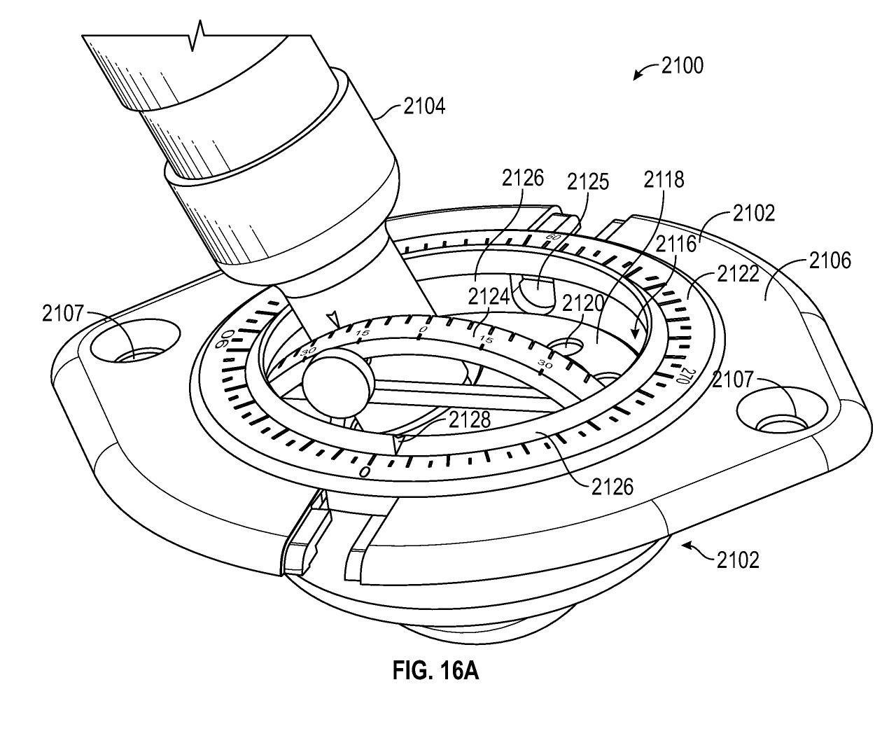

FIGS. 15A, 15B, and 15C show various views of an example of the base 104 in which at least one of: (1) the instrument exit portal 212 (in the cap 206); or (2) the instrument exit portal 214 (extending laterally across the flange 114), can optionally include a user-attachable, user-detachable, or user-attachable and user-detachable detent or restraint, such as a biocompatible clip 1502. The clip 1502 can be sized, shaped, or otherwise configured to help constrain or secure an instrument, such as a lead or catheter 702. The clip 1502 can provide an interference fit about the lead or catheter 702, such as to more securely anchor the lead or catheter 702 at the instrument exit portal 214.

In an illustrative example, the clip 1502 can include a trunk portion 1503, such as in a medial direction toward the center of the base 104. From the trunk portion 1503, a pair of legs 1505A-B can extend outward, such as in a lateral direction out from the center of the base 104. The clip 1502 can include one or more snap-fit or other engagement features 1506, such as can be located at opposing sides of the trunk portion 1503. The one or more engagement features 1506 can engage corresponding one or more mating or reciprocal snap-fit or other engagement features in the base 104 or the cap 206. The clip 1502 can fit within the instrument exit portal 212, 214 of the base 104 or the cap 206, such as flush to a face of a corresponding one thereof. In an example, such inserting of the clip 1502 into an instrument exit portal 212, 214 can push the legs 1505A-B toward each other, such as to compressively secure therebetween the lead or catheter 702 or other such instrument.

In an example, once the lead or catheter 702 is implanted, before the cap 206 is placed upon the base 104, the clip 1502 can be snap-fitted into one of the instrument exit portals 212, 214. Using the clip 1502 can permit the other passageways (e.g., the instrument exit portals 212, 214) to be larger, more gentle, or more forgiving. In this way, when the clip 1502 is not present, the lead or catheter 702 can be more easily removed by pulling it out. Such pulling can involve the user exerting a pulling force on a more proximal location of the lead or catheter 702, e.g., away from the clip 1502.

A tether 1504 optionally can be attached to the clip 1502. The tether 1504 can be routed subcutaneously, along with the lead or catheter 702, for a desired distance or to a desired location. Beyond this desired subcutaneous routing distance, the lead or catheter 702 can emerge proximally out from under the skin, such as together with a proximal portion of the tether 1504. By pulling on an exposed proximal portion of the tether 1504, a user can use the tether 1504 as a “ripcord,” such as to remotely release the clip 1502 from the base 104 or the cap 206. Such releasing of the clip 1502 from the base 104 or the cap 206 can allow the lead or catheter 702 to move freely at the instrument exit portals 212, 214, which can allow convenient user extraction or removal of the lead or catheter 702 by pulling on the proximal end of the tether 1504.

The tether 1504 can optionally include a coiled or slack portion between the clip 1502 and the proximal end of the tether 1504, such as at a subcutaneous location, if desired. This can help guard against accidental release of the clip 1502, e.g., by an accidental minor tug on the tether 1504 that does not exceed a pull distance needed to release the slack in the tether 1504. In an example, the tether 1504 can be omitted, and the lead or catheter 702 can itself be used by the user as a ripcord to release the clip 1502 from the base 104, such as by suturing, clipping, or otherwise affixing the clip 1502 to the lead or catheter 702, such as at the instrument exit portal 212, 214.

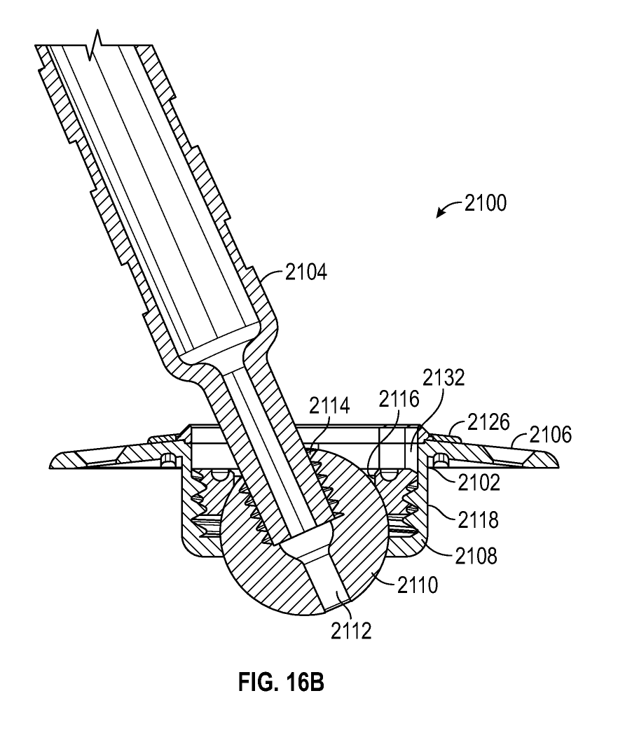

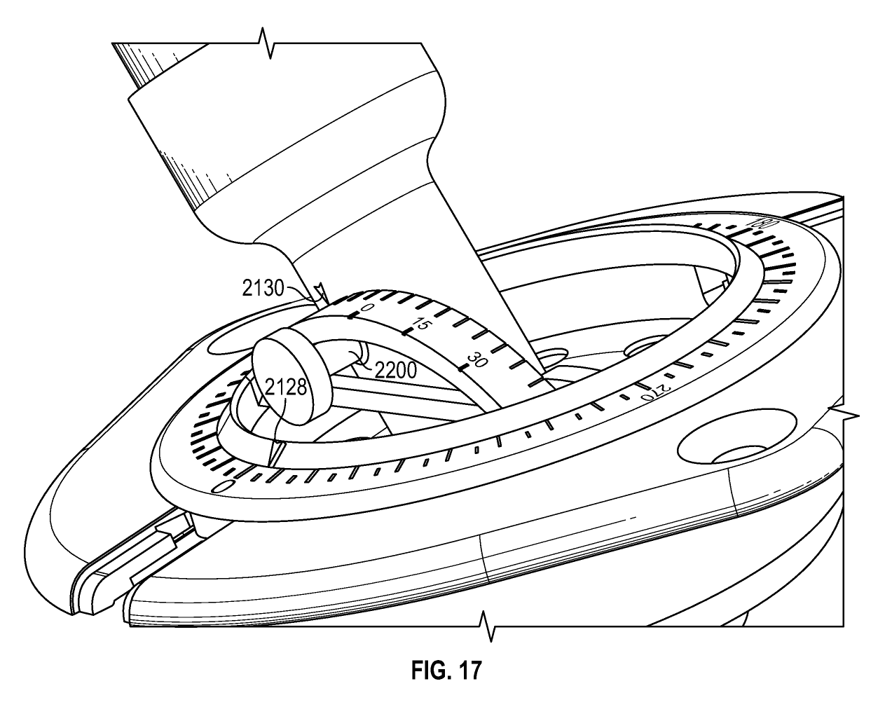

FIGS. 16A (plan view) and 16B (cross-section view) show an example of a body-mounted trajectory guide, such as a skull-mounted trajectory guide 2100, that can be mounted onto a subject's skull such as about a desired skull entry portal, such as a burr hole, such as for guiding an instrument through the skull entry portal and toward a desired path into the subject's brain. The trajectory guide 2100 can include a base 2102 and an adjustably positionable instrument guide stem 2104, which can be hollow or can include a lumen such as to allow passage of the guided instrument or other instrument therethrough. The base 2102 can include a low-profile flange 2106 that can extend laterally outward from a socket 2108. The flange 2106 can be secured to the subject's skull, such as via bone screws respectively extending through bone screw passages 2107 on the flange 2106. The socket 2108 can be sized and shaped such that it can fit within the burr hole or other desired skull entry portal. The socket 2108 can be sized and shaped to accept a spherical or other ball 2110. The ball 2110 can have a central pivot point within the socket 108 below a bottom surface of the flange 2106, such as when the flange 2106 is seated against the skull about the burr hole. The ball 2110 can include a passage 2112 therethrough. The passage 2112 can be sized and shaped to permit the instrument being guided to pass therethrough. The passage 2112 can include a proximal portion that can provide a receptacle 2114 that can be sized and shaped to receive or engage a distal end of the guide stem 2104, such as by threads or one or more other engagement features that can be respectively included within the passage or elsewhere on the ball 2110 or on the distal portion of the guide stem 2104.

The socket 2108 can provide a proximally-facing internal receptacle 2116, at least a portion of which can be sized and shaped to accept a spherical or other ball 2110. The ball 2110 can be pivotably seated against a bottom portion of the receptacle 2116, such as with a central pivot point of the ball 2110 being located below a bottom-facing surface of the flange 2106. A portion of the ball 2110 can protrude at least partially below the bottom portion of the receptacle 2116, such as into the burr hole or other entry portal such as when the flange 2106 is seated on the skull. A retainer ring 2118 can be engaged into the receptacle 2116 of the socket 2108 such as to secure the ball 2110 into a desired position such as to provide the desired trajectory for introducing the instrument through the guide stem 2105, the entry portal, or to a desired location within the subject. The retainer ring 2118 can include one or more threads or other engagement features such as to permit engagement of the retainer ring 2118 into the socket 2108, such as in a manner that can seat against a proximal portion of the ball 2110 to secure the ball 2110 in a desired pivoted position such as after the ball 2110 has been pivotably adjusted by an end-user (or an automated or semi-automated control device) such as by manipulating the guide stem 2104 to pivot the ball 2110. The retainer ring 2118 can also include one or more proximally-accessible engagement features 2120, such as can be engaged from above by a tool or otherwise, such as to thread the retainer ring 2118 into the receptacle 2116 of the socket 2108, such as to secure the ball 2110.

The base 2102 can also include a rotational alignment indicator, such as can be provided by one or more indicia or features on a rotational alignment ring 2122. For example, the rotational alignment indicia can indicate degrees between 0 and 360 degrees about the circular rotational alignment ring 2122. The rotational alignment ring 2122 can be integrally formed with or fixed to the flange 2106, or alternatively can be separately formed and rotatably engaged to the flange 2106 such as to be rotated into a desired position, such as to align a desired rotational alignment indicator (e.g., 0 degrees) with a desired direction with respect to the subject (e.g., the anterior-posterior (A-P) direction or other desired direction), even if the base 2102 is not aligned in any particular direction when mounted on to the subject's skull.

The base 2102 can also include a pivot sweep guide arch 2124, such as can extend proximally from a pivot sweep guide arch ring 2126. The pivot sweep guide arch 2124 can include a pivot sweep alignment indicator, such as can be provided by indicia or features on the pivot sweep guide arch 2124 that can indicate a degree of tilt, such as in a forward or reverse direction from a vertical zero point. The pivot sweep guide arch ring 2126 can include an arrow or other alignment indicator 2128. The pivot sweep guide arch ring 2126 can be rotated with respect to the rotational alignment ring 2122, and the alignment indicator 2128 can be read against the indicia on the rotational alignment ring 2122, such as to provide an indication of rotational alignment.

The pivot sweep guide arch 2124 can advantageously constrain movement of the ball 2110 such that the guide stem 2104 travels against the pivot sweep guide arch 2124 when it is tilted by the end-user or a control device. In an example, such arching constraint of the guide stem 2104 can be provided by a pin or thumbscrew 2200 or other feature on the guide stem 2104 that travels against the pivot sweep guide arch 2124, such as along the underside of the pivot sweep guide arch 2124, in such a manner that the guide stem 2104 is constrained against the pivot sweep guide arch 2124 during tilting. The thumbscrew 2200 can be tightened, such as to secure the guide stem 2104 at a desired forward or desired tilt, which can be read by an arrow or other alignment indicator against the indicia on the pivot sweep guide arch 2124. The thumbscrew 2200 can alternatively be removed, and the desired tilt (and rotation) of the guide stem 2104 can be secured such as by tightening the retainer ring 2118 against the ball 2110. For example, the location of the pivot sweep guide arch 2124 can be laterally offset away from a center diameter of the pivot sweep guide arch ring 2126, such as to allow space for a tool to be inserted within the pivot sweep guide arch ring 2126, such as to engage one or more of the engagement features 2120 on the retainer ring 2118 such as to allow the retainer ring 2118 to be secured against the ball 2110.

A disc or other spacer 2132 can optionally be located between the retainer ring 2118 and the pivot sweep guide arch ring 2126. The spacer 2132 can include a center cutout such as to permit access to the engagement features 2120 of the retainer ring 2118 by a tool for tightening or loosening the retainer ring 2118. The spacer 2122 can also include one or more exit portals 2124, such as can be sized and shaped and located to permit a leadwire, catheter, or other instrument to laterally exit the base 2102, such as via the exit portals 2124 or similar exit portals in the socket 2108 or flange 2106 portions of the base 2102.

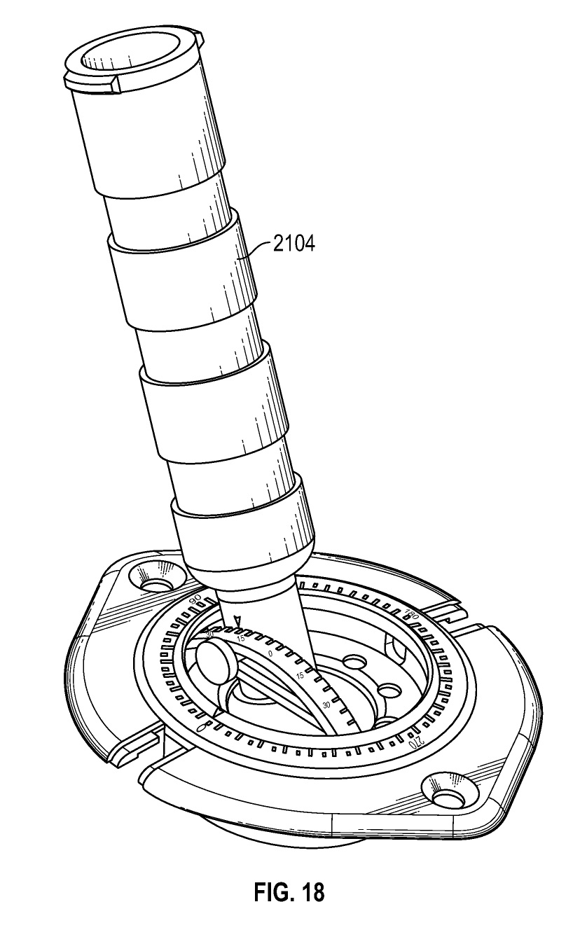

FIG. 18 shows another view of the trajectory guide 100 with a proximal portion of an example of the guide stem 104 shown.

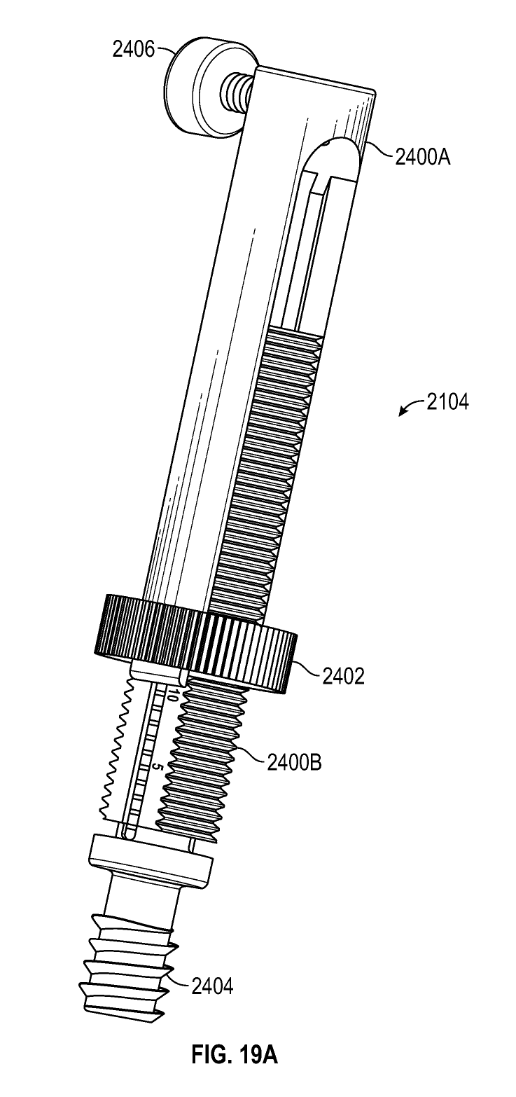

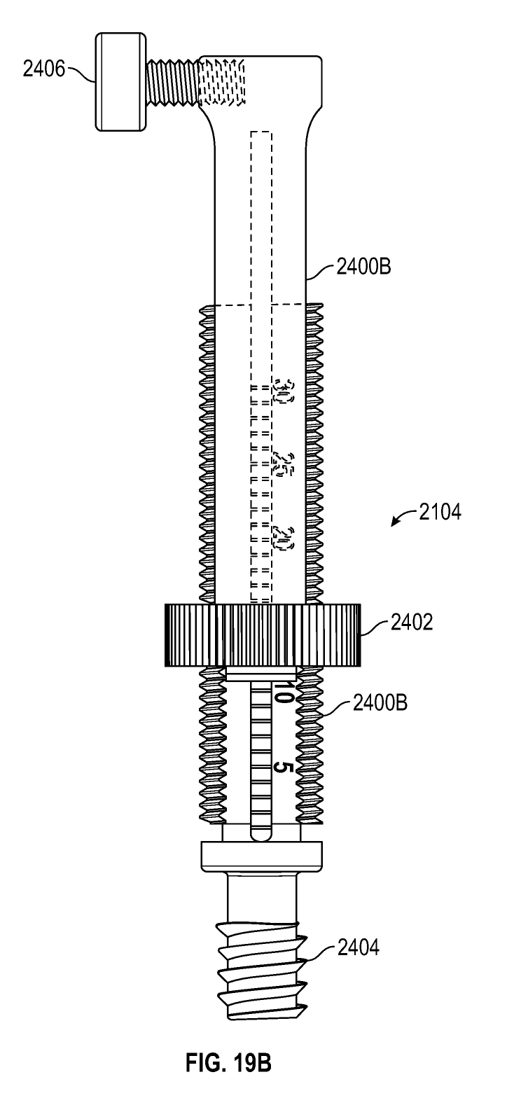

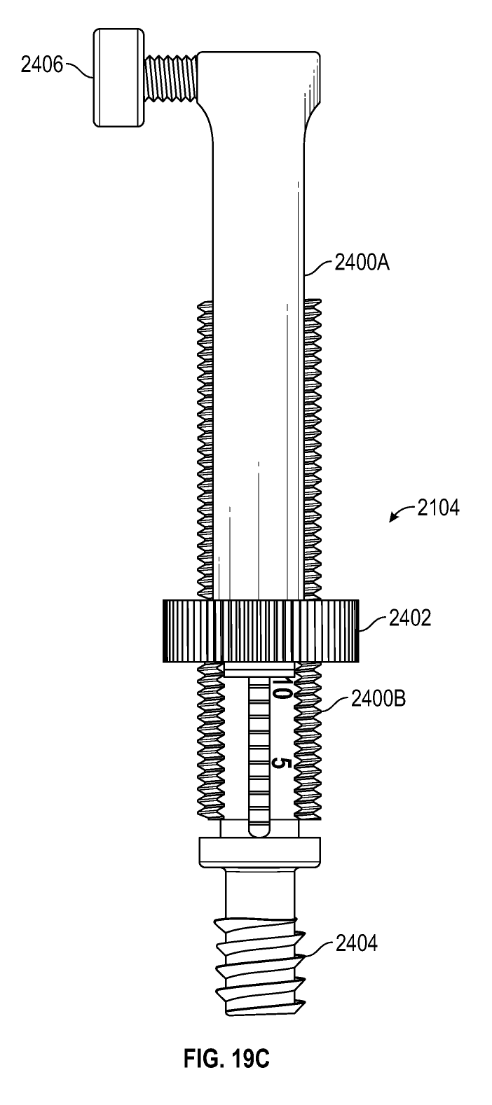

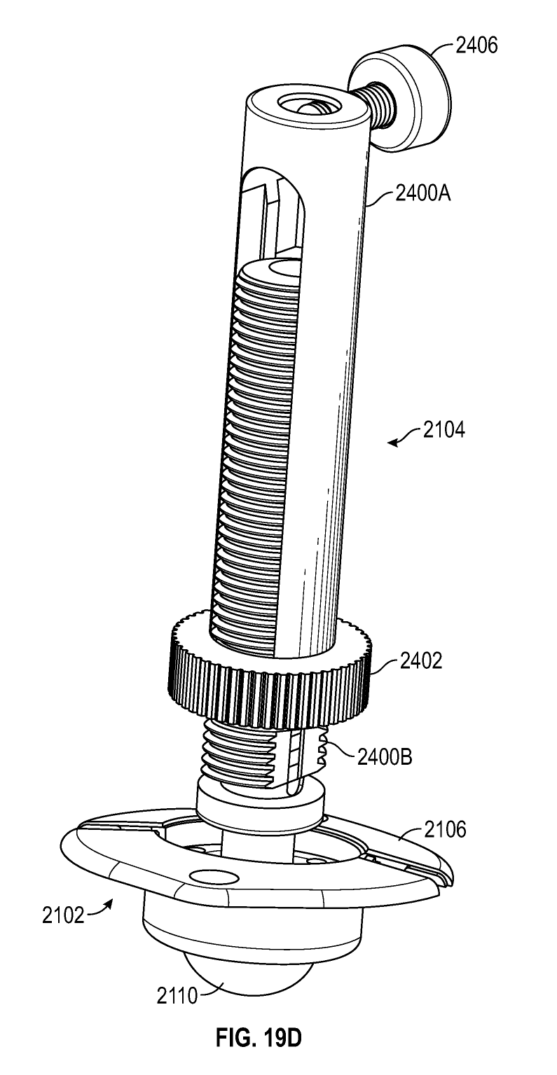

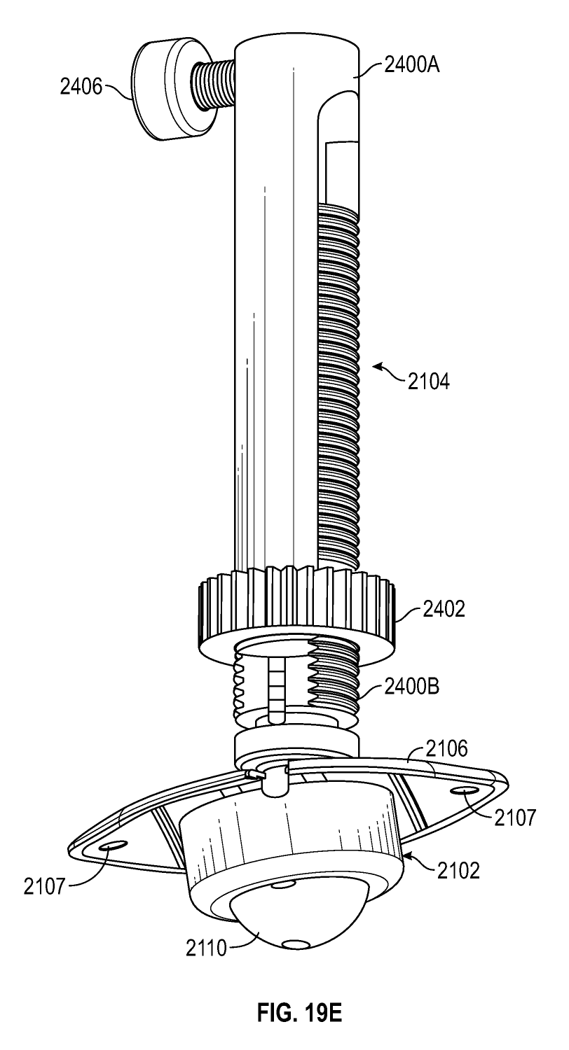

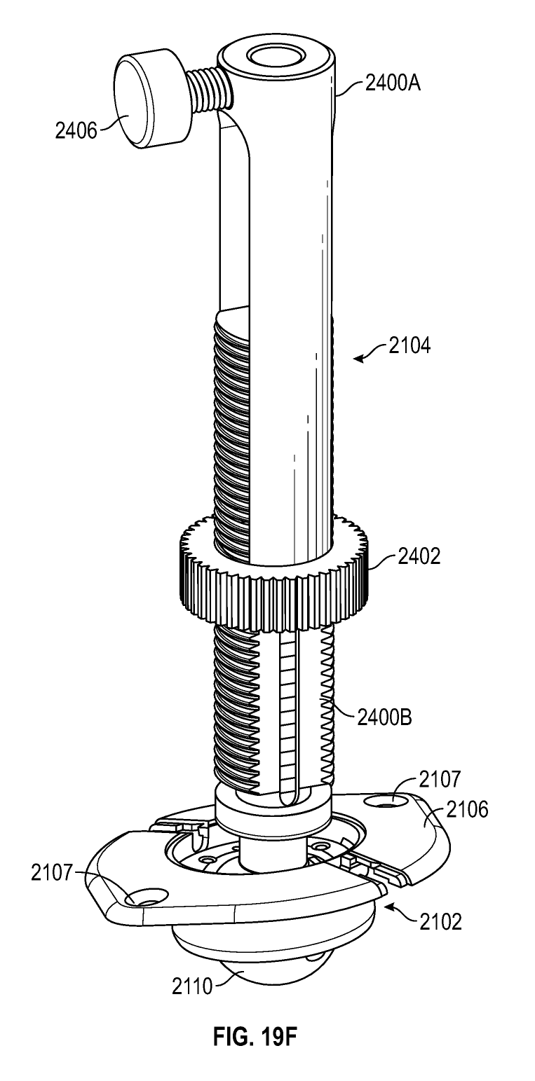

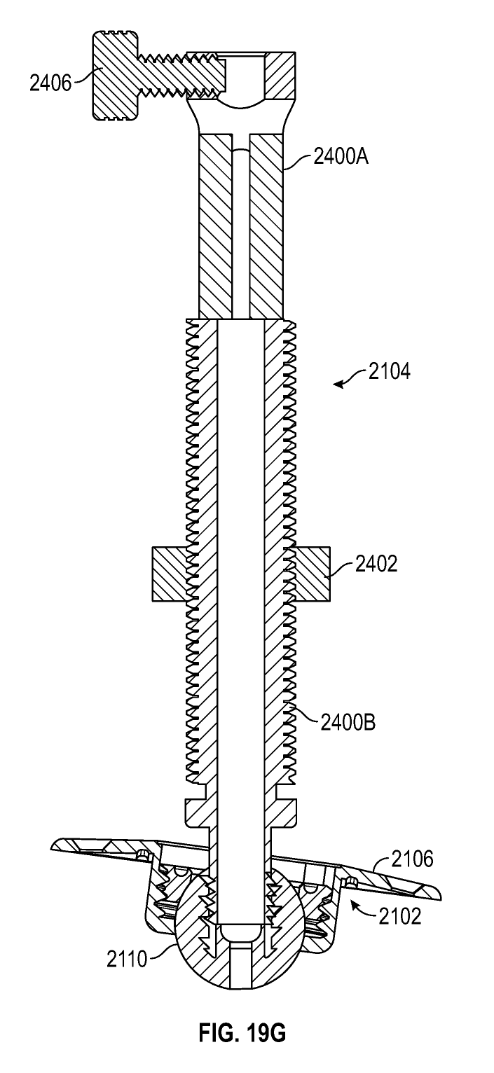

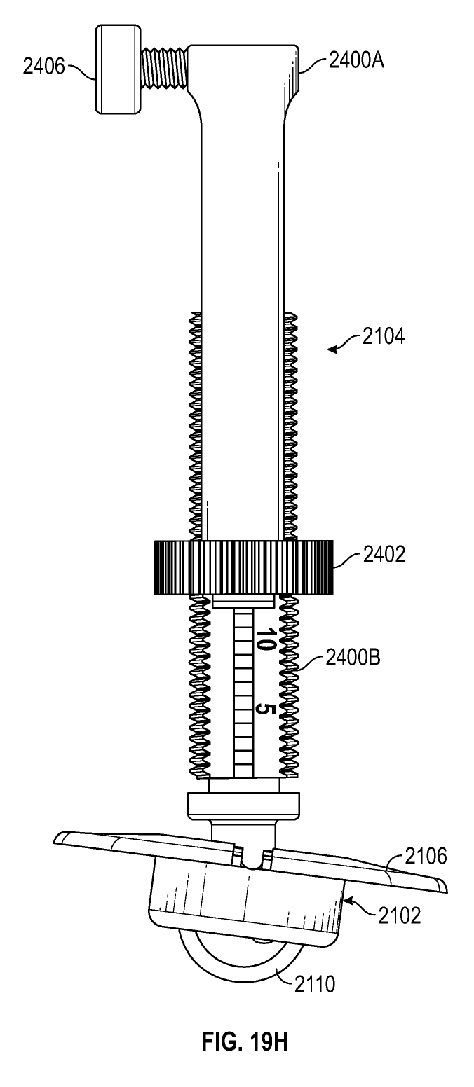

FIGS. 19A, 19B, 19C, 19D, 19E, 19F, 19G, 19H, and 19I show an example of a guide stem 2104 that can include a “Z-Direction” height adjustment, such as for providing a desired height or length of the guide stem 2104 or for mounting one or more other components to the guide stem 2104 at a desired Z-Direction height. For example, the guide stem 2104 can include two components, such as an inner shaft or sleeve 2400A and an outer sleeve 2400B, such as can be threadably coupled with respect to each other, such as to provide a desired height of the guide stem 2104. In an example, a threaded thumb wheel 2402 can be engaged to one of the inner or outer sleeves 2400A-B and turned by the user such as to threadably adjust the longitudinal position of the sleeves 2400A-B with respect to each other. Height indicia can be provide on one of the inner or outer sleeves 2400A-B and read against an end or other indicator on the other of the inner or outer sleeves 2400A-B, such as to provide an indication of the then-current value of the Z-Direction height adjustment.

The guide stem 2104 can include distal threads 2404, such as for being threaded into the ball 2110, such as explained above. The guide stem 2104 can include a thumb screw 2406, such as at a proximal end of the guide stem 2104, such as to secure one or more components to the guide stem, such as before or after Z-height adjustment of the guide stem 2104.

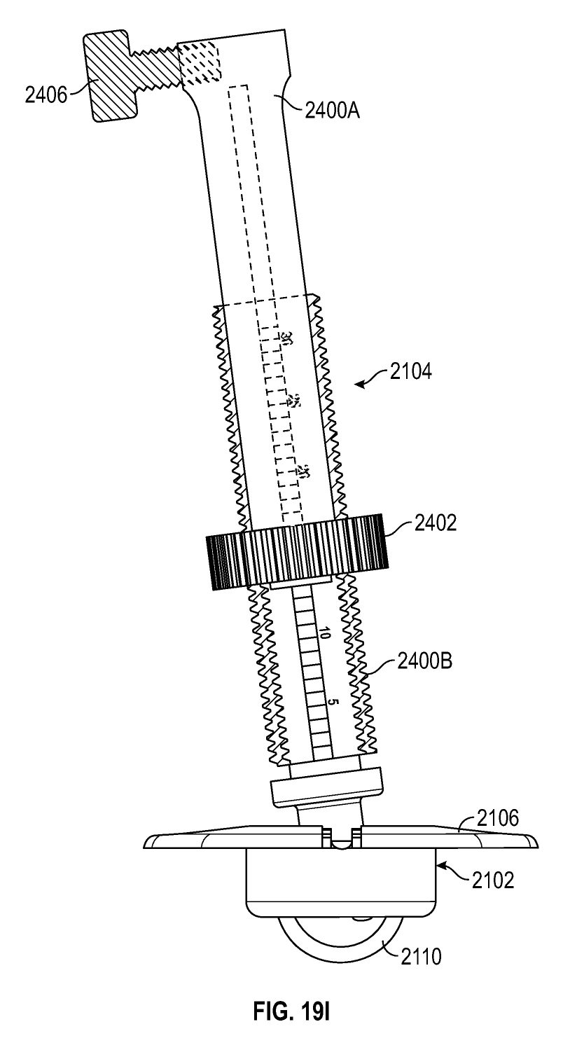

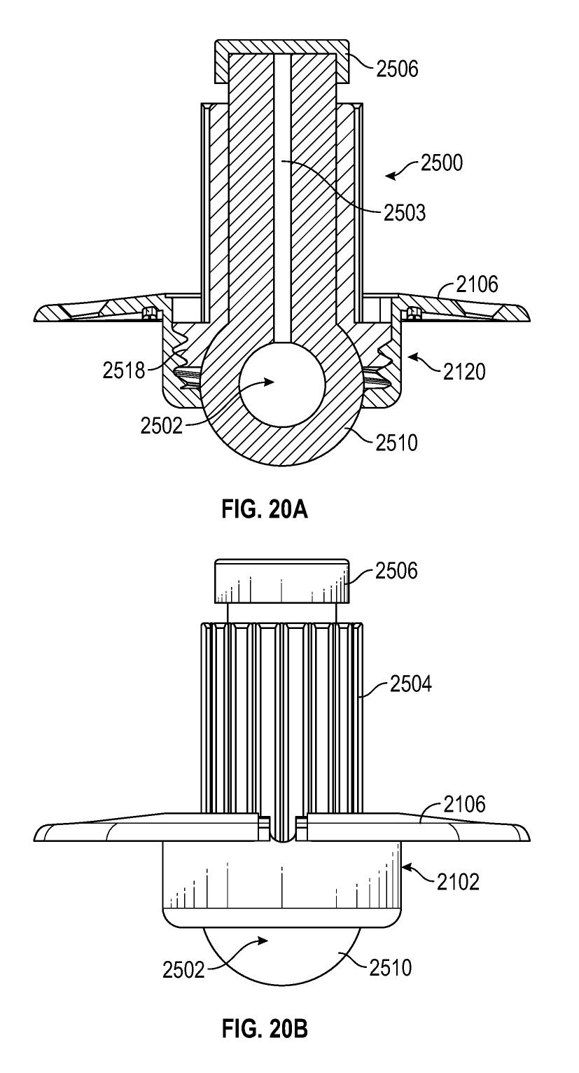

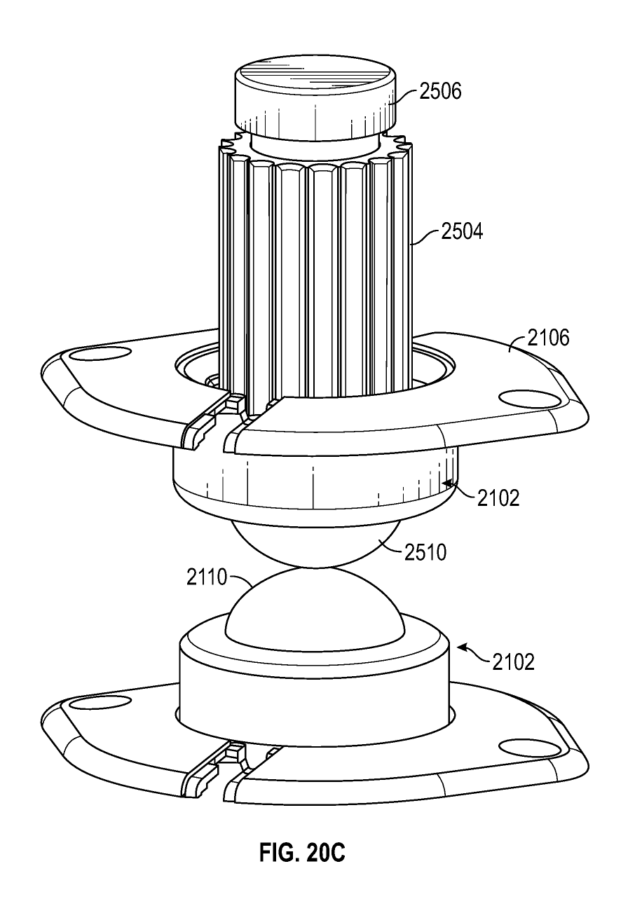

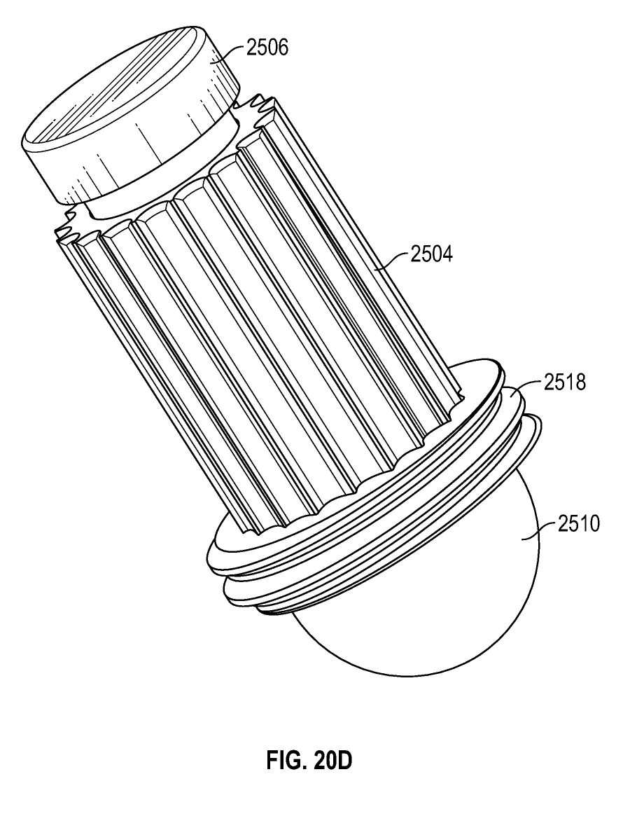

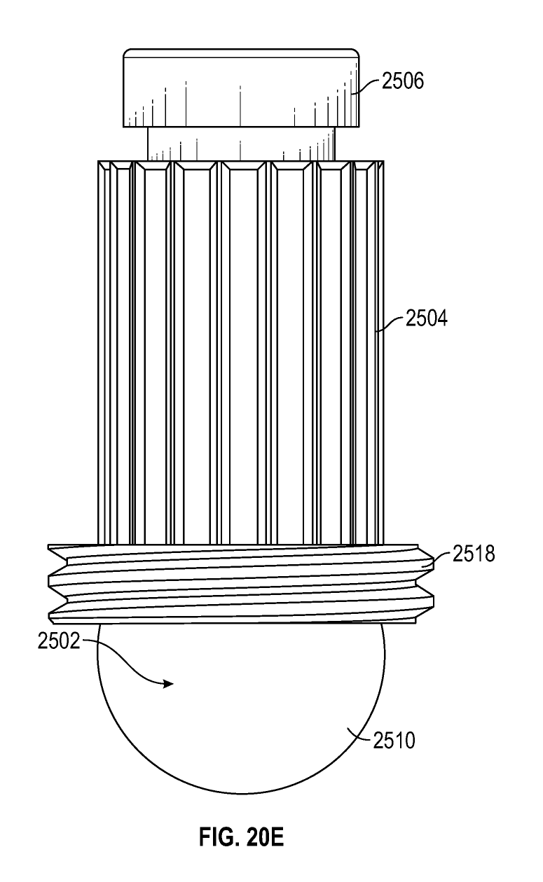

FIGS. 20A, 20B, 20C, 20D, and 20E show various views of an imaging fiducial stem 2500 that can be used together with the base 2102, such as instead of one or more of the guide stem 2104, the ball 2110, and the retainer ring 2118, such as during an imaging session by a medical imaging modality, such as magnetic resonance imaging (MR or MRI), computed tomography (CT), or positron emission tomography (PET). The imaging fiducial stem 2500 can include one or more imageable fiducial components that show up on the selected one or more imaging systems, such as with sufficient contrast to allow medical diagnosis or treatment. For example, the imaging fiducial stem 2500 can include a fiducial marker that can clearly demarcate on an imaging display the central pivot point of the pivoting ball 2110 of the trajectory guide 2100. Optionally, one or more other aspects of the trajectory guide 2100 (e.g., the ball 2110, the base 2102, the guide stem 2104, etc.) can additionally or alternatively be demarcated on an imaging display, such as by one or more other contrast-enhanced or other fiducial markers. When an imaging scan has been created that marks the pivot point, the imaging fiducial stem 2500 can be removed and the subject can either be (1) “stitched up” and sent home for further medical procedures on another day, or (2) sent on for further medical procedures on the same day.

For example, for MRI, the imaging fiducial stem 2500 can include one or more of an imageable (e.g., fluid-filled with a MR contrast agent) ball central pivot location fiducial marker 2502 and an optional imageable (e.g., fluid filled with a contrast agent) longitudinal instrument guide trajectory fiducial marker 2503, both of which can be configured to provide good contrast on an MRI image, such as from the materials of the trajectory guide 2100, the subject's skull and brain tissue, or both. The fiducial markers 2502 and 2503 can be formed from the same unitary chamber within the imaging fiducial stem 2500, such as to accept a fluid contrast agent. A seal or cap 2506 can be located at a fluid intake port to the chamber to seal and confine the fluid contrast agent within the imaging fiducial stem 2500.

The imaging fiducial stem 2500 can be sized and shaped and otherwise configured to mimic the alignment guide stem 2104, such that when the imaging fiducial stem 2500 is inserted in and fully threaded into the receptacle 2116 of the socket 2108, the center of the fiducial marker 2502 is at the same location that the center of the ball 2110 would be if the imaging fiducial stem 2500 were replaced by the ball 2110, the retainer 2118, and the guide stem 2104. Similarly, the fiducial marker 2503 will be at the same location that the instrument-guiding trajectory provided by the center passage of the guide stem 2104 would be if the imaging fiducial stem 2500 were replaced by the ball 2110, the retainer 2118, and the guide stem 2104.

In an example, the imaging fiducial stem 2500 can include (such as in a single component) a ball portion 2510 (e.g., mimicking ball 2110), a threaded retainer 2518 that can be integrally formed with or otherwise attached to the ball portion 2510 (e.g., mimicking the retainer 2118), and a guide stem 2504 (e.g., mimicking the guide stem 2104) that can be integrally formed with or otherwise attached to the threaded retainer 2518 and the ball portion 2510).

In this way, the imaging fiducial stem 2500 can be used, such as during preoperative or intraoperative imaging session, to plan the trajectory of the instrument insertion under MR imaging guidance, and the guide stem 2104, ball 2110, and retainer 2118 can be used later, such as to obtain the same desired alignment using the information from the imaging session.

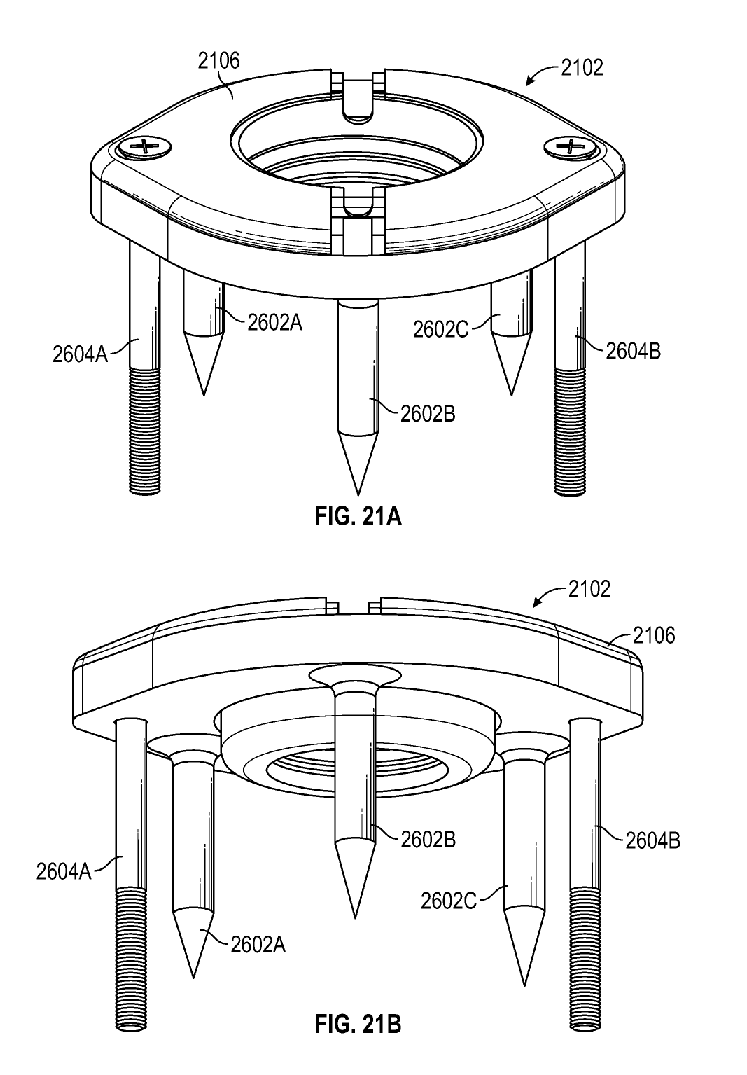

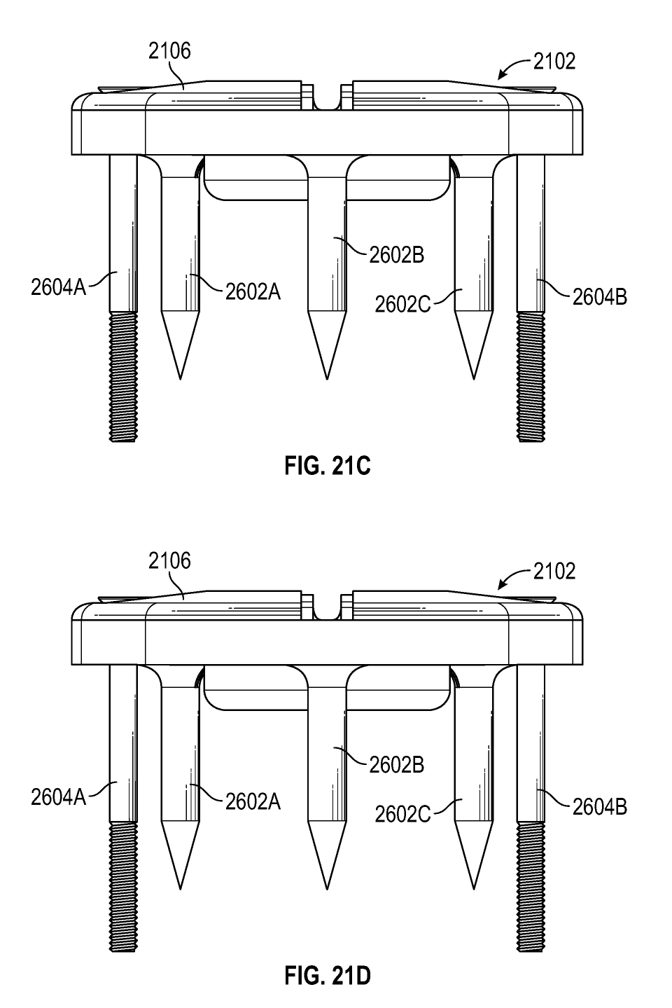

FIGS. 21A, 21B, 21C, and 21D show an example in which the base 2102 of the trajectory guide 2100 can optionally include three or more legs 2602, such as to permit the base 2102 to be raised above the burr hole or other entry portal. The legs 2602 can include sharp tips at their distal ends, away from the base 2102, such as to help plant the legs 2602 against the subject's skull and to inhibit or prevent sliding relative to the subject's skull. One or more bone screws 2604 can be used to secure the raised base 2102 to the subject's skull at the desired location. The one or more bone screws can be passed through one or more screw hole openings 2107 in the flange 2106 of the base 2102. The raised base 2102 such as shown in FIGS. 20A-20D can help provide an ability to align the trajectory first, and then drill (e.g., by extending a drill bit through the center lumen of the guide stem 2104) an “on-trajectory” hole through the subject's skull to provide an entry portal. The resulting “on-trajectory” hole can be smaller than a typical (e.g., 14 millimeter) burr hole.

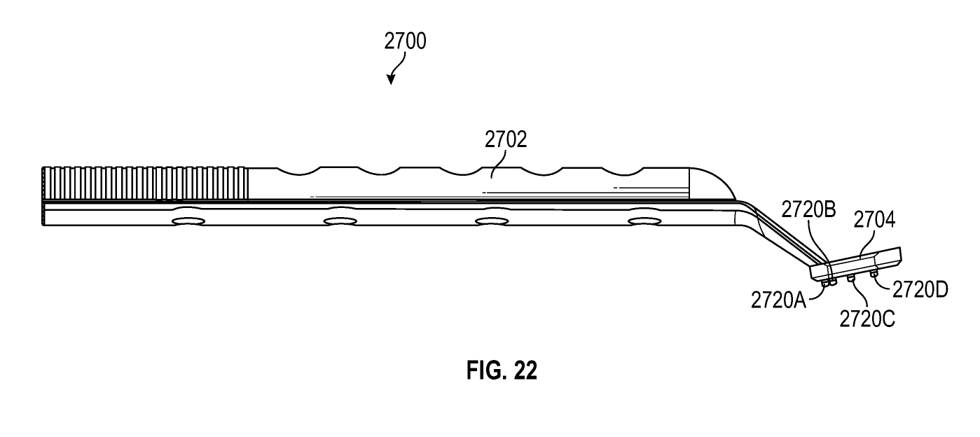

FIG. 22 shows an example of a wrench or other tool 2700 that can be used to tighten the retainer ring 2118 to secure the ball 2110 in a desired position, which, in turn, can provide the desired instrument trajectory via the guide stem 2104 that can be attached to the ball 2110. The tool 2700 can include a handle 2702 and a working distal portion 2702 that can be sized, shaped, or otherwise configured to be placed flat against the retainer ring 2118 with one or more engagement features 2720 (such as protrusions) engaged with one or more corresponding engagement features 2120 in the retainer ring 2118. An outer circumference of the working distal portion 2702 can be sized to fit within the receptacle 2116 of the socket 2108, such as to permit turning the working distal portion 2702 to thread the retainer ring 2118 into the receptacle 2116 of the socket 2108.

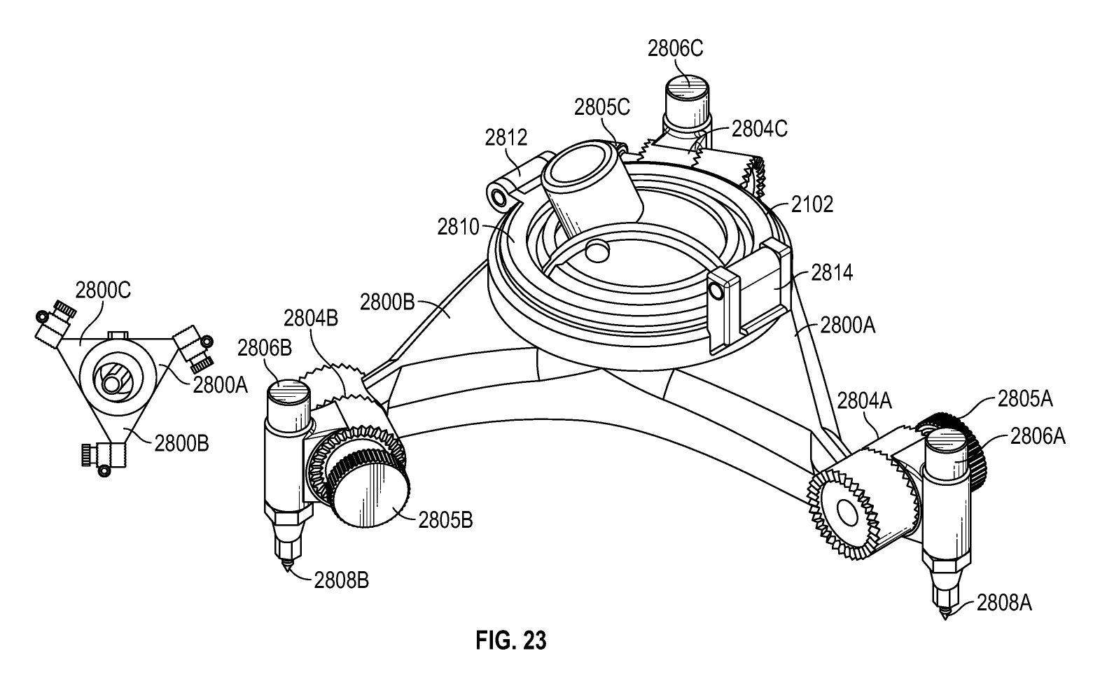

FIG. 23 shows an example in which the skull mounted trajectory guide 2100 (e.g., such as shown in FIG. 16A, 16B, 17, or 18) in which the base 2102 can be raised above the skull, along with the ball 2110 and the socket 2108. In an example, this can include providing a tripod, a single leg or other support, or a plurality of legs 2800, such as can extend laterally outward or down toward the skull, or both. In FIG. 23, the legs 2800A-C can include or be coupled to feet 2802A-C, such as at the peripherally distal portions of the legs 2800A-C. The feet 2802A-C can be angularly or otherwise adjustable, such as by including a locking serrated joint 2804A-C, such as a Hirth joint or Hirth coupling, such as can be locked using a thumbscrew to draw opposing serrated disks of the joint together. The feet 2802A-C can include a fixed or height-adjustable peg 2806A-C on a peripheral portion of the respective joint 2804A-C. The pegs 2806A-C can be height adjustable, such as by being threadable with a receptacle on the peripheral portion of the respective joint 2804A-C, such as using thumbscrews 2805A-C for the pegs 2806A-C or using another height adjustment technique. The pegs 2806A-C can include sharp threaded bone screw distal tips 2808A-C, such as to secure the pegs 2806A-C and, in turn, the entire base 2102, to the skull.

The position of the rotating or swiveling ball 2110 can be secured within the socket 2108, such as by using a clamping retaining ring 2810. The retaining ring 2810 can be can be pressed downward such as to clamp over the ball 2110. A hinge 2812 can couple the retaining ring 2810 to the base 2102. A user-engageable and user-disenageable clasp 2814 can secure the retaining ring 2810 to the base 2102. In an example, the raised base 2102 or the raised socket 2108, or both, can include a light-emitting diode (LED) or other lamp, such as on the underside toward the skull, such as to provide light that can be directed toward the burr hole or other desired location of the skull underneath the raised base 2102 or the raised socket 2108. A local or remote power supply can be provided such as to provide electrical power to the lamp such as via a wired connection to the lamp.

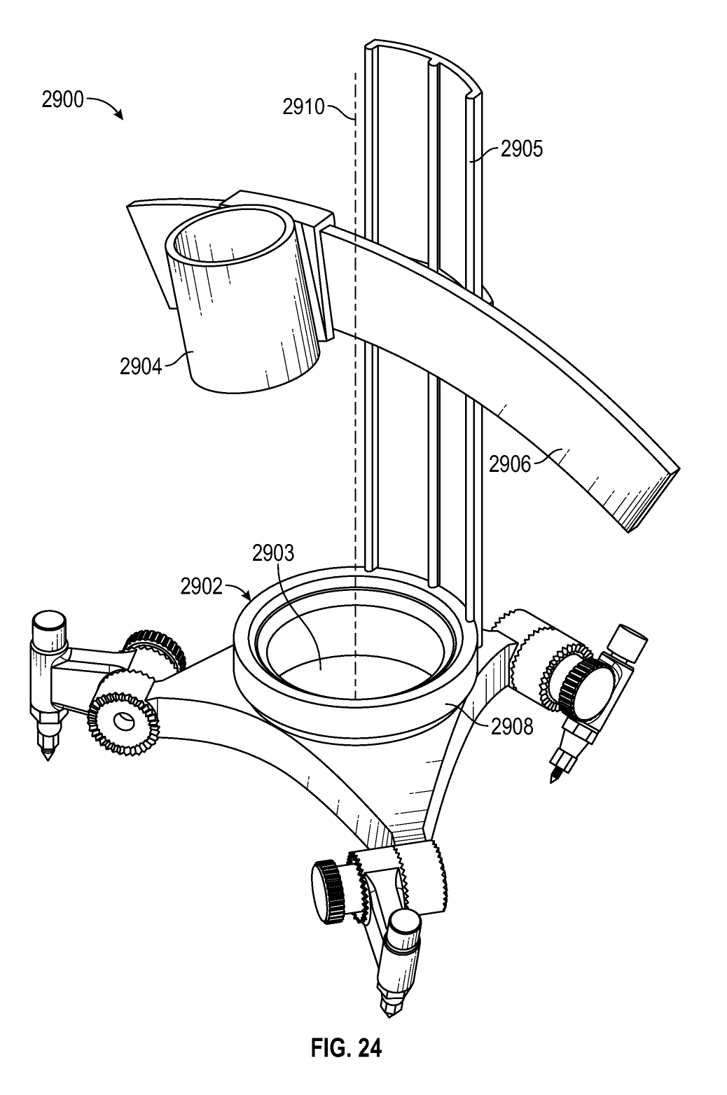

FIG. 24 shows an example of a “target-centered” skull mounted trajectory guide 2900 in which the base 2902 can be raised above the skull, such as described herein, such as with respect to FIG. 24. In an example, the base 2902 need not include a ball and socket to establish the trajectory, but instead can include a sufficiently large opening providing a portal 2903 through the base 2902 and a movable aiming barrel 2904. The aiming barrel 2904 can include a contrast-enhanced imageable fiducial marker and can be movable along an arc 2906. The arc 2906 can be raised above the base 2902 such as by one or more posts 2905, such as can extend from a swivel 2908 coupling the one or more posts 2905 to the base 2902 such as to allow 360 degree swiveling rotation about an axis 2910 defined longitudinally through a center of the portal 2903 in the base 2902. The swivel can include respectively engaging rings, such as can also include bearings, if desired. Such swiveling can move the aiming barrel 2904 to alter an approach direction of a trajectory provided by the aiming barrel 2904. Moving the aiming barrel 2904 along the arc 2906 can vary the angle of the trajectory through the opening 2903 and toward a common target location within the skull beyond a burr hole in the skull. The one or more posts 2905 can include a guide slot or track. The guide slot or track can allow a vertical (“Z” direction) height location of the arc 2906 to be adjusted upward or downward by the user. One or more thumbscrews or other securing devices can be used by the user to secure the arc to the one or more posts 2905 such as adjustably at the desired height above the base 2902. A lower “Z” height setting can correspond to a deeper target location beyond the skull. A higher “Z” height setting can correspond to a shallower target location beyond the skull. The arc 2906 can be made rotatable around a center axis through the opening 2903 in the base 2902. This can include coupling the one or more posts 2905 to the base 2902 via a rotation ring that can engage the base 2902 and can rotate about the base 2902 and be secured in a desired rotational orientation, such as by a thumbscrew or other securing apparatus.

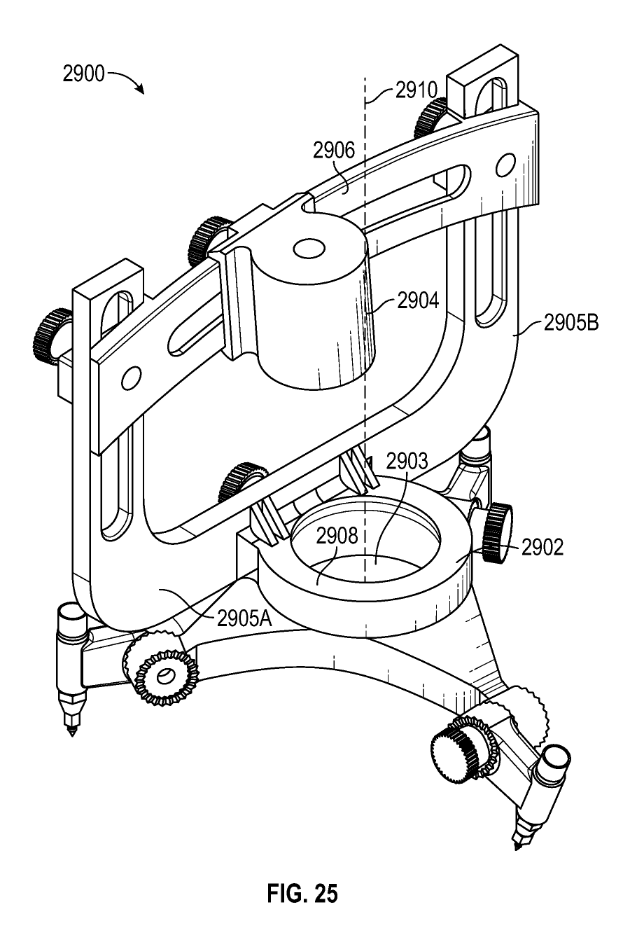

FIG. 25 shows an example of a “target-centered” skull mounted trajectory guide 2900, such as described herein such as above with respect to FIG. 24, but in which the arc 2906 can extend between two posts 2905A-B, such as to provide additional mounting stability for the arc 2906.

The target centered trajectory guide alignment method can use initial alignment and placement using the fluid filled MRI/CT mechanism described herein, an image guided surgery system, such as the Medtronic Stealth® or Treon®, or a stereotactic head frame. The “Z” or “depth to target” can be the same for all procedures and can be set at initial alignment. One potential advantage of a target centered apparatus and method is that the entry point can be shifted such as to avoid one or more cortical vessels without changing the location of the target. One or more of the movable components (e.g., the barrel 2904, the arc 2906, the ball 2110, the swivel 2908, the retainer ring 2810, guide stem 2104, pivot sweep guide arch ring 2126, threaded thumb wheel 2402, etc.) can be robotically driven, such as by using a microactuator controlled by a microcontroller circuit or the like.

In an example, a method of establishing a trajectory using a target-centered embodiment, such as described herein such as with respect to FIGS. 24 and 25, can include: (1) mounting the base 2902 to the subject's skull, such as about a burr hole in the skull; (2) performing imaging, such as by using an imageable fiducial marker such as described herein; (3) establishing the desired Z height, such as by adjusting the height of the arc 2906 to give a desired distance to the target; and (4) rotating the arc 2906 about a center axis extending vertically through the center of the opening 2903, and/or sweeping the trajectory angle, such as by moving the barrel 2904 along the arc 2906, such as to determine a desired entry point, such as while maintaining a trajectory toward a desired fixed target location within the skull.

Although FIG. 23 showed an example of a raised base in combination with a ball-and-socket trajectory guide configuration, and FIGS. 24 and 25 showed examples of a raised base in combination with a “target centered” (e.g., arc and barrel) trajectory guide configuration, the raised base can also be used to provide a combined configuration, such as in which can provide a ball-and-socket trajectory guide and a target-centered trajectory guide. This can include a providing a base with a user-attachable and user-detachable ball-and-socket. When the user removes the ball-and-socket, such component removal can provide the opening 2903 for a target-centered trajectory guide, other user-attachable and detachable components of which (e.g., the one or more posts 2905, the barrel 2904, etc.) can then be attached by the user.

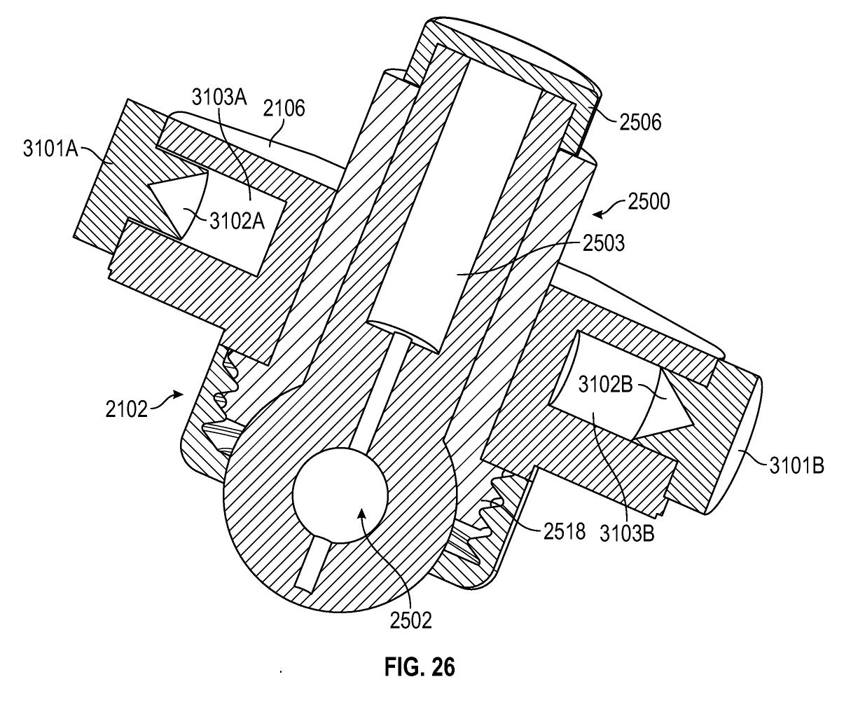

FIG. 26 shows an example in which the trajectory guide 2100 can include certain components having imageable fiducial markers, similar to that shown and described above with respect to FIG. 20A. In the example of FIG. 26, the flange 2106 can include MR, CT, or other imageable fiducial markers 21101A-B, such can be located on opposing lateral edges of the flange 2106. The fiducial markers 21101A-B can be sized, shaped, or otherwise configured to fit into corresponding or mating receptacles 21103A-B on the opposing lateral sides of the flange 2106. The user can visually align (e.g., with or without using imaging information) such fiducial markers 21101A-B in an anterior-posterior (A-P) or other desired direction, which can then be verified or compensated for during pre-operative or intraoperative imaging. The fiducial markers 21101A-B can be fluid-filled with a contrast agent. The fiducial markers 21101A-B can include recessed portions or can otherwise be shaped or configured so as to respectively provide “arrows” 21102A-B or another imageably visualizable indication of directionality. In the example of FIG. 26, in addition to being able to provide a fluid volume 2502 that indicates the pivot point, the fiducial markers 21101A-B can provide imageable volumes on opposing lateral portions of the trajectory guide flange 2106 that allow indication of anterior and posterior directions to appear in a distinguishable manner on images provided by the imaging modality. This can allow workstation software to compensate for any possible misalignment of the actual placement of the trajectory guide with respect to the actual anterior and posterior directions, such as can be determined using software processing of images obtained using the imaging modality.

FIGS. 27A-B shows an example, similar to that shown and described with respect to FIG. 23, in which the trajectory guide 2100 can include a base 2102 that can include an adjustable stage 21201. The adjustable stage 21201 can allow the user or controller device to adjust a location of the socket 2108 (and the ball 2110 carried therein), such as within an adjustment plane. In an example, the stage 21201 can provide angle polar offset, such as by using a circular disk stage 21201 that can rotate within the base 2102, such as a full 360 degrees about a longitudinal center axis defined by a correspondingly sized circular receptacle of the base 2102 that receives the circular disk stage 21201. The circular disk stage 21201 can be secured in a desired angular orientation, such as by a thumbscrew 21202. The circular disk stage 21201 can also include a lateral (side-to-side) translatable sub-stage 21203, such as can allow the socket 2108 (and the ball 2110 carried therein) to be laterally translated and repositioned by a user or by a controller device, such as by manipulating a thumbscrew 21204 engaging both the base 2102 and the sub-stage 21203. FIG. 27B shows an example of an x-y stage 21205, which can similarly be placed within a corresponding receptacle in the base 2102, such as to allow translation of the socket 2108 back-and-forth in an x-direction and also back-and-forth in a y-direction. Either of the adjustable stages 21201 or 21205 can be used with the base 2102 without the socket 2108 and ball 2110, if desired. In such a case, socket 2108 as shown in FIGS. 27A-B can be replaced by an appropriately-sized lumen to guide therethrough a correspondingly sized instrument, such as a catheter, a recording or stimulation electrode, or the like. For example, in a microelectrode recording application (e.g., for epilepsy diagnosis or characterization), a polar-offset or x-y adjustable stage for a trajectory guidance lumen (without requiring a socket 2108 and ball 2110) can be useful such as for mapping an x-y grid of locations on a surface of the brain, such as along parallel but laterally offset trajectories.

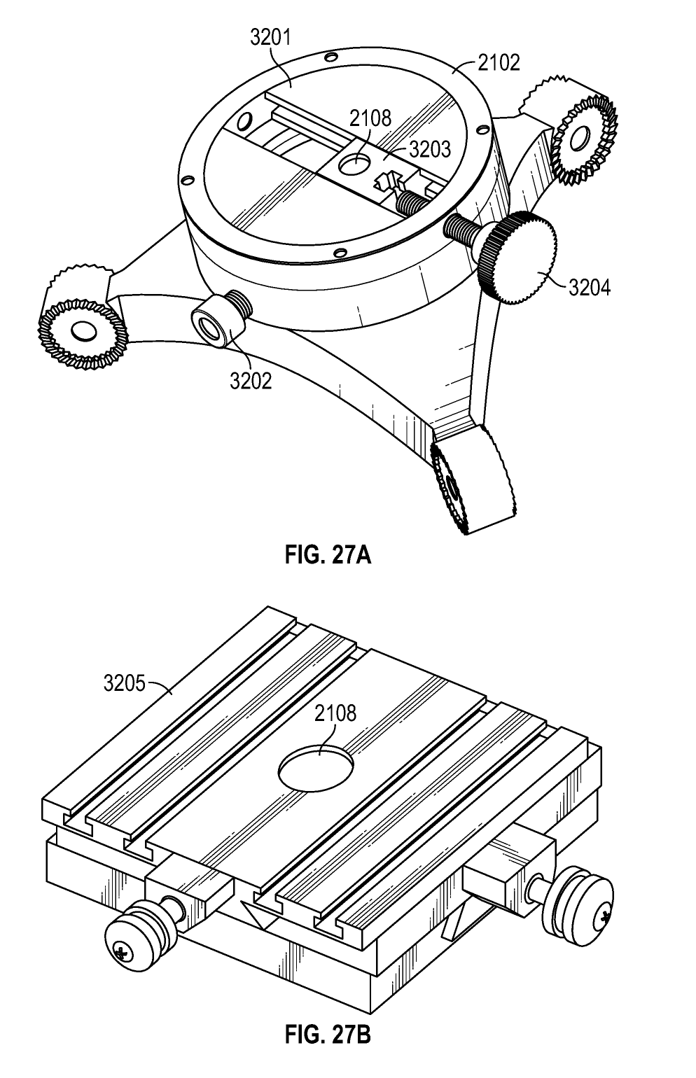

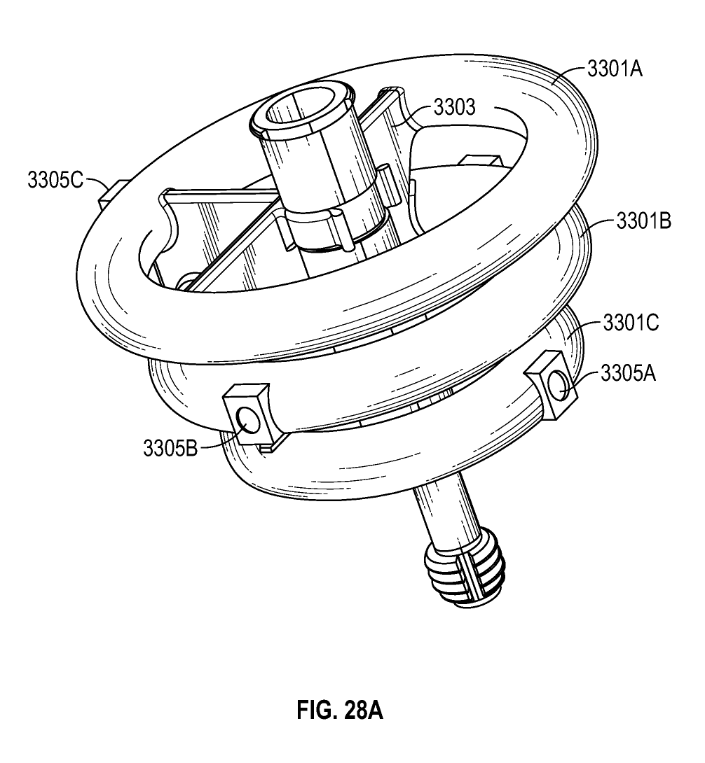

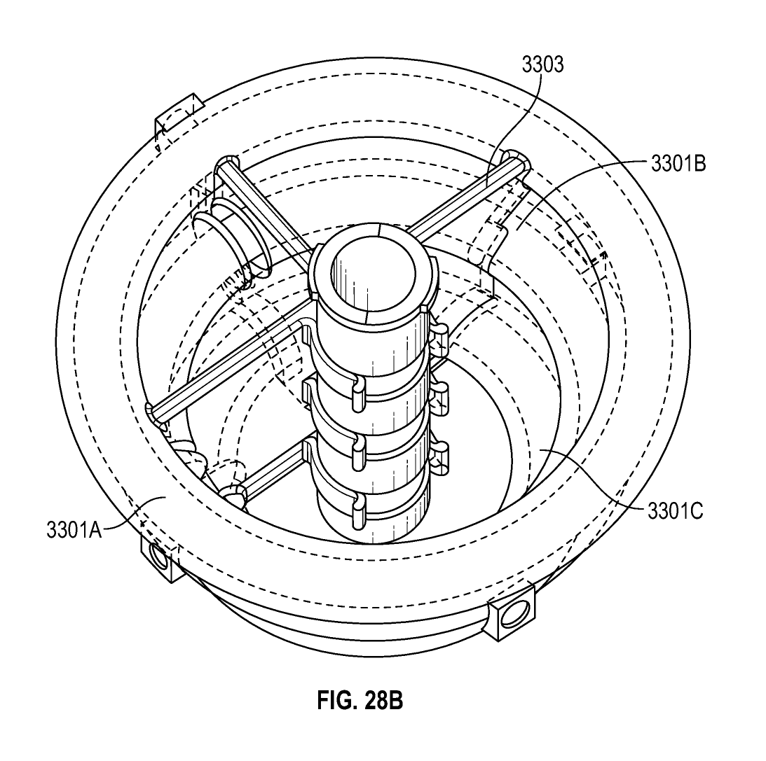

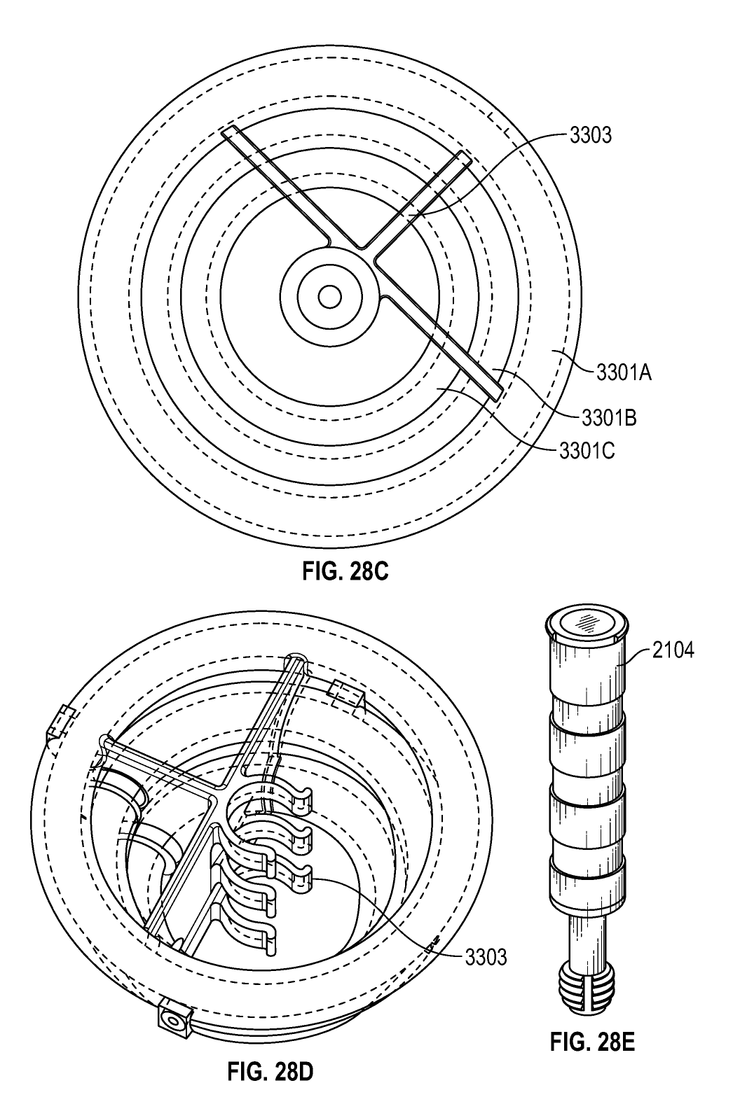

FIGS. 28A, 28B, 28C, 28D, and 28E show an example of two or more concentric ring imageable fiducial marker rings 21301A-C, such as can be clipped or snapped onto or otherwise affixed to a proximal portion of the guide stem 2104, such as using a clip-on rack 21303 that can include multiple clips that clip onto respective recessed portions of the guide stem 2104. The rack 21303 can arrange the locations of the rings 21301A-C with respect to each other. The rings 21301A-C can be affixed to the rack 21303 in concentric alignment with each other. The rings 21301A-C can be in a tapered arrangement such that they are progressively smaller in diameter. For example, the ring 21301C can be smaller in diameter than the ring 21301B, which, in turn, can be smaller in diameter than the ring 21301A. The outer diameter of a smaller ring can be smaller than an inner diameter of the next larger ring such that, when directly viewed concentrically looking from a proximal end of the guide stem 2104 toward a distal end of the guide stem 2104, a small gap between the rings can be seen.

The rings 21301A-C can be fluid-filled, e.g., with an imageable contrast agent to enhance their visibility on a desired imaging modality, such as via fluid-fill ports 21305A-C. The fluid fill ports 21305 can be located at desired locations about the circumference of the set of rings 21301, such as at 0 degrees, 90 degrees, and 180 degrees, as shown in FIG. 28D. The fluid fill ports 21305 can include an additional volume of the contrast agent such that the fluid fill ports 21305 themselves can be visible on an imaging modality, and the orientation of the fluid fill ports 21305 can thereby be used as fiducial marker indicators. The rings 21301 can be progressively smaller in diameter to visually show on an image produced by an imaging modality the “TARGET CENTERED” when aligned in an orthogonal view of the trajectory. The rings 21301 and rack 21303 can be rotated together such that the fluid fill ports 21305 (visible on the image by their contrast agent fluid) can provide reference for anterior-posterior and medial-lateral directions.

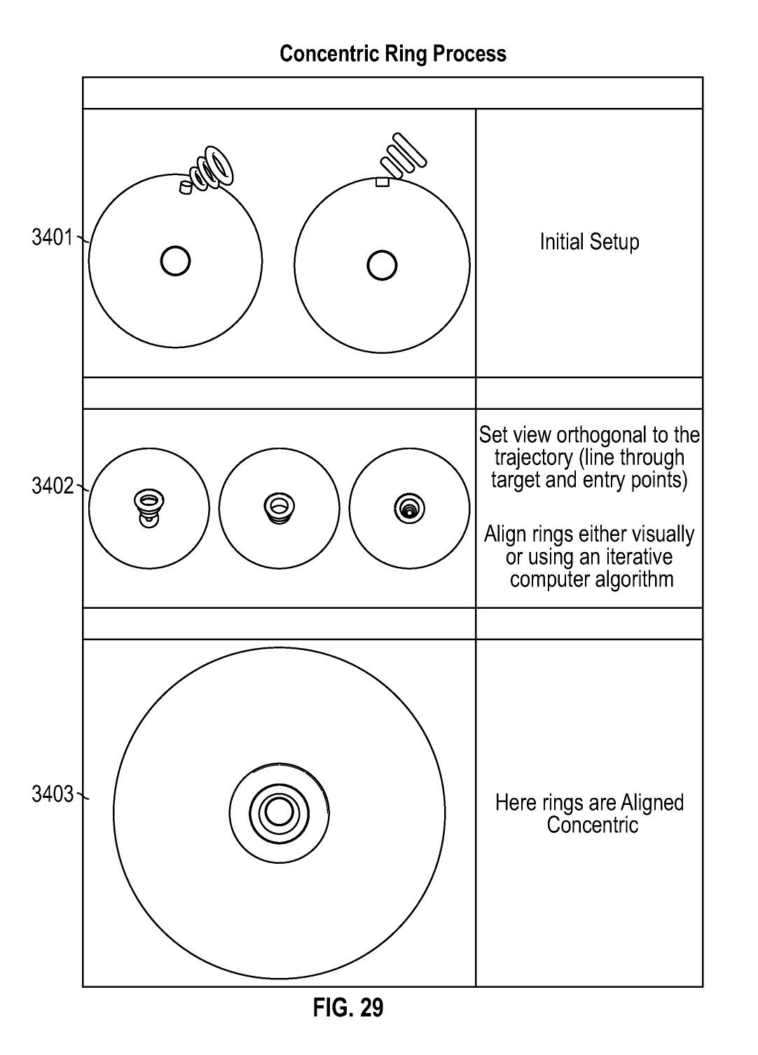

FIG. 29 is a diagram illustrating an example of trajectory guide alignment using the tapered arrangement of concentric rings 21301A-C. At 21401, an example of an arrangement of the rings 21301 during initial setup is shown, together with the entry point, the target, and the trajectory. At 21402, a view on the imaging modality is set orthogonal to the trajectory (line through the target and entry points). The guide stem 2104 is then adjusted (manually or using a controller device) until the rings are concentrically aligned, as shown at 21403, such as with the gaps between the progressively smaller rings visible to indicate alignment.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. A trajectory guide apparatus, comprising:
a base, configured to be able to be capable of being affixed substantially at a burr hole in a human or animal subject;
a pivotably adjustable instrument guide, coupled to and pivotably adjustable with respect to the base, the instrument guide defining a longitudinal open passage therethrough defining an adjustable instrument trajectory; and
a first set of one or more fiducial markers, disposed in and commonly defining a first plane extending orthogonal to the trajectory, and defining a first common centroid on the first plane at an intersection with the trajectory within the open passage.

2. The apparatus of claim 1, comprising:
a platform that is user-attachable to and user-detachable from the proximal end of the elongate guide stem, the platform comprising the first set of one or more fiducial markers, disposed in and commonly defining a first plane extending orthogonal to the trajectory, and defining the first common centroid on the first plane at the intersection with the trajectory within the open passage, the platform comprising an opening configured to allow the instrument trajectory therethrough.

3. The apparatus of claim 1, further comprising a second set of one or more fiducial markers, disposed in and commonly defining a second plane extending orthogonal to the trajectory and spaced apart from the first plane, and defining a second common centroid on the second plane at an intersection with the trajectory within the open passage.

4. The apparatus of claim 3, wherein the first and second sets of fiducial markers respectively include spaced-apart rings that are each concentric to the open passage and the trajectory.

5. The apparatus of claim 4, wherein the rings are different from each other in at least one of an inner ring diameter and an outer ring diameter.

6. The apparatus of claim 3, further comprising a third set of one or more fiducial markers, disposed in and commonly defining a third plane extending orthogonal to the trajectory and spaced apart from the first plane and from the second plane, and defining a third common centroid on the third plane at an intersection with the trajectory within the open passage.

7. The apparatus of claim 6, wherein the first, second, and third sets of fiducial markers respectively include spaced-apart rings that are each concentric to the open passage and the trajectory.

8. The apparatus of claim 4, wherein the first and second sets of fiducial markers respectively include spaced-apart triangular arrangements of fiducials that are each concentric to the open passage and the trajectory.

9. A method of adjusting a trajectory of a pivotably adjustable trajectory guide that was previously affixed to a subject at an entry portal, the trajectory guide providing a longitudinal passage defining an instrument trajectory to be adjusted to be directed to align with a desired trajectory to a target location within the subject, the trajectory guide including a first set of one or more fiducial markers, disposed in and commonly defining a first plane extending orthogonal to the trajectory, and defining a first common centroid on the first plane at an intersection with the trajectory within the open passage, the method comprising:
visualizing a view in a plane orthogonal to the desired trajectory;
pivotably adjusting the passage until the first centroid aligns with the desired trajectory;
securing the passage with the first centroid aligned with the desired trajectory; and
passing an instrument through the secured passage toward the target.

10. The method of claim 9, comprising further visualizing the view in the plane orthogonal to the desired trajectory to confirm that the first centroid remains aligned with the desired trajectory while the instrument remains passed through the passage toward the target.

11. The method of claim 9, wherein the visualizing comprises using a CT or MRI imaging modality.

12. The method of claim 9, comprising determining the first centroid using image analysis software.

13. The method of claim 9, wherein the trajectory guide further includes a second set of one or more fiducial markers, disposed in and commonly defining a second plane extending orthogonal to the trajectory, and defining a second common centroid on the second plane at an intersection with the trajectory within the open passage wherein the first and second sets of fiducial markers include first and second rings respectively defining the first and second centroids, the first and second rings concentric to and spaced apart from each other, wherein the method comprises:
pivotably adjusting the passage until the rings are aligned to each other in a viewing plane orthogonal to the desired trajectory.

14. The method of claim 9, wherein pivotably adjusting the passage comprises:
adjusting a rotational orientation of the passage with respect to an anterior-posterior orientation of the subject; and
adjusting an angular orientation of the passage until the first centroid aligns with the desired trajectory.

15. A method of determining a trajectory of a pivotably adjustable trajectory guide that was previously affixed to a subject at an entry portal, the trajectory guide providing a longitudinal passage defining an instrument trajectory to be adjusted to be directed to align with a desired trajectory to a target location within the subject, the trajectory guide including a first set of one or more fiducial markers, disposed in and commonly defining a first plane extending orthogonal to the trajectory, and offset from and defining a first common centroid on the first plane at an intersection with the trajectory within the open passage, the method comprising:
passing an instrument through the open passage toward the target; and
with the instrument located within the open passage, visualizing a view in a plane orthogonal to the desired trajectory to determine whether the first centroid aligns with the desired trajectory using the first set of one or more fiducial markers that are offset from the passage in the first plane.

16. The method of claim 15, further comprising:
pivotably adjusting the passage until the first centroid aligns with the desired trajectory; and
securing the passage with the first centroid aligned with the desired trajectory.

17. The method of claim 15, wherein the visualizing comprises using a CT or MRI imaging modality.

18. The method of claim 15, comprising determining the first centroid using image analyzing software.

19. The method of claim 15, wherein the trajectory guide further includes a second set of one or more fiducial markers, disposed in and commonly defining a second plane extending orthogonal to the trajectory, and defining a second common centroid on the second plane at an intersection with the trajectory within the open passage wherein the first and second sets of fiducial markers include first and second rings respectively defining the first and second centroids, the first and second rings concentric to and spaced apart from each other, wherein the method comprises:
pivotably adjusting the passage until the rings are aligned to each other in a viewing plane orthogonal to the desired trajectory.

20. The method of claim 19, wherein pivotably adjusting the passage comprises:
adjusting a rotational orientation of the passage with respect to an anterior-posterior orientation of the subject; and
adjusting at least one of the rotational orientation of the passage or an angular orientation of the passage until the first centroid aligns with the desired trajectory.

21. The method of claim 19, comprising:
determining a desired angular orientation of the passage using a difference between the rotational orientation of the passage and the anterior-posterior orientation of the subject; and
using the determined desired angular orientation for the adjusting the angular orientation of the passage.
